(19) United States
(12) Patent Application Publication
(10) Pub. No.: US 2026/0069785 A1
(43) Pub. Date: Mar. 12, 2026

(54) INJECTION DEVICE WITH ADD-ON DEVICE

(71) Applicant: Sanofi, Paris (FR)

(72) Inventors: Stefan Alt, Frankfurt am Main (DE); Michael Helmer, Frankfurt am Main (DE)

(21) Appl. No.: 19/107,410

(22) PCT Filed: Aug. 28, 2023
(86) PCT No.: PCT/EP2023/073462

§ 371 Date: Feb. 27, 2025

(30) Foreign Application Priority Data
Aug. 30, 2022 (EP) 22315199.4

(51) Int. Cl. A61M 5/24; A61M 5/315; G06K 7/10

(52) U.S. Cl. CPC A61M 5/2422; A61M 5/31568; G06K 7/10297; G06K 7/10366; A61M 2005/2496; A61M 2205/14; A61M 2205/3379; A61M 2205/3584; A61M 2205/52; A61M 2205/6054

(57) ABSTRACT

Disclosed is an injection device for injecting of a dose of a medicament that includes a housing having a body and a container part configured to accommodate a medicament container and detachably connectable to the body, a drive mechanism arranged in the body and operable to expel the dose of the medicament from the medicament container, a machine-readable identification on or inside one of the body and the container part, and a detector arrangement operable to detect a mechanical connection between the body and the container part. The machine-readable identification is electrically connected to the detector arrangement, is readable by an electronic module of an add-on device connectable to the injection device and is indicative of the mechanical connection between the body and the container part.

### CROSS-REFERENCE TO RELATED APPLICATIONS

The present application is the national stage entry of International Patent Application No.

PCT/EP2023/073462, filed on Aug. 28, 2023, and claims priority to European Application No. EP 22315199.4, filed on Aug. 30, 2022, the disclosures of which are incorporated herein by reference.

### TECHNICAL FIELD

The present disclosure relates to the field of injection devices, in particular to injection devices, such as a pen-type injectors usable with dedicated add-on devices. In another aspect the disclosure relates to an injection system comprising an injection device and an add-on device.

In further aspects the disclosure relates to a method of monitoring operation of injection device.

### BACKGROUND

Drug delivery devices for setting and dispensing a single or multiple doses of a liquid medicament are as such well-known in the art. Generally, such devices have substantially a similar purpose as that of an ordinary syringe.

Drug delivery devices, such as pen-type injectors, have to meet a number of user-specific requirements. For instance, with patients suffering chronic diseases, such as diabetes, the patient may be physically infirm and may also have impaired vision. Suitable drug delivery devices especially intended for home medication therefore need to be robust in construction and should be easy to use. Furthermore, manipulation and general handling of the device and its components should be intelligible and easy understandable. Such injection devices should provide setting and subsequent dispensing of a dose of a medicament of equal or variable size. Moreover, a dose setting as well as a dose dispensing procedure must be easy to operate and has to be unambiguous.

A patient suffering from a particular disease may require a certain amount of a medicament to either be injected via a pen-type injection syringe.

Some drug delivery or injection devices provide selecting of a dose of a medicament of variable size and injecting a dose previously set. Other injection devices provide setting and dispensing of a fixed dose. Here, the amount of medicament that should be injected in accordance to a given prescription schedule is always the same and does not change or cannot be changed over time.

Some injection devices are implemented as reusable injection devices offering a user to replace a medicament container, such as a cartridge. Other injection devices are implemented as a disposable injection device. With disposable injection devices it is intended to discard the entirety of the injection device when the content, i.e. the medicament, has been used up.

In order to control and to supervise administering of medication conducted by users or patients themselves it is beneficial to assist the user by making use of an external electronic device, such as a mobile electronic device, e.g. implemented as a smartphone, a tablet computer, or a smart watch. A software application provided on such external electronic devices may interact with the user and may provide instructions or recommendations to the user of how to correctly use the injection device.

In order to control and to supervise administering of medication conducted by users or patients themselves it is desirable to provide an automated detecting and logging of a repeated and regular use of the drug delivery device. A rather automated recording of doses injected by a user would offer a significant advantage over a manual dose logging in terms of security and convenience.

There exist numerous add-on devices configured for use with injection devices that offer an electronic detection and monitoring of repeated dose injection procedures.

Typically, such add-on devices or auxiliary devices can be detachably connected to an injection device. An add-on device typically comprises a detector or detector arrangement operable to detect a date and/or time when the user sets or injects a dose of the medicament. Some add-on devices also provide a quantitative measurement of a size of a dose currently set or dispensed. Some add-on devices are intended for use with a series of injection devices. This may particularly apply with disposable injection devices, that are intended to become discarded after use or after the medicament located therein has been used up.

Generally, there exists a variety of different injection devices, which may be equipped with different drugs or medicaments but are generally operable to be used with one and the same type of an add-on device. While it is common practice to characterize or to label injection devices and/or medicaments of different types and/or of different concentration by a clear and distinct label or identification a detachable connection the detachable connection with an add-on device may impose a disadvantage, in particular when a label or a characteristic portion of the injection device is covered by the add-on device when attached to the injection device.

With reusable injection devices that provide replacement of a medicament container it is desirable to reset the drive mechanism. When such reusable injection devices are used with an add-on device it may be also required to conduct a reset operation or some kind of initialization process with the add-on device as well. Where the add-on device is configured to log or to track a residual amount of medicament left in a cartridge it may be also required to reset a data logging procedure and/or to initialize a new tracking procedure with the add-on device when a medicament container of the injection device is replaced.

In view of this it is desirable to provide improvements to an injection device and/or to an add-on device in order to simplify the process of device handling in the course of replacing a medicament container. Moreover, logging or monitoring of repeated use of the injection device should be simplified. Moreover, patient safety should be enhanced.

### SUMMARY

In one aspect the present disclosure relates to an injection device for injecting of a dose of a medicament. The injection device comprises a housing. The housing comprises a body and a container part. The container part is configured to accommodate a medicament container. The container part is further detachably connectable to the body. The injection device further comprises a drive mechanism arranged in the body and being operable to expel or to withdraw the dose of the medicament from the medicament container.

The injection device further comprises a machine-readable identification on or inside one of the body and the container part. The injection device further comprises a detector arrangement on or inside one of the body and the container part. The detector arrangement is operable to detect a mechanical connection between the body and the container part. The machine-readable identification is electrically connected or electrically linked to the detector arrangement. It is readable by an electronic module of an add-on device, which add-on device is connectable, i.e. mechanically connectable to the injection device. The machine-readable identification is indicative of the mechanical connection between the body and the container part.

Typically, the injection device is implemented as a handheld injection device. It may comprise a pen-type injector. The injection device may be implemented all-mechanically. It may be void of any electrical or electronic components. The one and only electric or electronic component may be the machine-readable identification of the injection device and the detector arrangement. The machine-readable identification may be implemented as an electronic identifier. The machine-readable identification may comprise a passive wireless communication tag or an active wireless communication tag, such as a passive or active RFID tag, NFC tag or UWB tag. Insofar the injection device may be void of any active electric or electronic components.

The machine-readable identification may comprise an electronic identifier, which is electronically readable by the add-on device. The machine-readable identification may comprise a radiofrequency transceiver of passive or active type. With some examples the machine-readable identification is implemented as one of a RFID tag, a NFC tag or UWB tag. With some examples the machine-readable identification is electronically readable by a transceiver and/or by a respective electronic module of the add-on device. Hence, the machine-readable identification is configured to establish a wireless communication link with the add-on device thus allowing to read data or information stored in the machine-readable identification.

The electrical connection or the electronic link between the machine-readable identification and the detector arrangement of the injection device is such, that a state of the detector arrangement can be electronically read or read out by the add-on device. The state or status of the detector arrangement is indicative of the mechanical connection between the body and the container part

Typically, the add-on device is configured for fastening to a proximal end of the body or drive mechanism of the injection device. With some examples the injection device may comprise a dial extension, which is subject to at least one of a rotational and a longitudinal displacement relative to the body of the injection device during or for setting of a dose and/or during or for injecting of a dose of the medicament. While the add-on device is e.g. exclusively attachable or fastenable to the proximal end of the injection device and hence to a respective proximal end of the body or drive mechanism of the injection device it may be and may always remain in the transmission range of the machine-readable identification of the injection device when and/or while attached thereto.

With some examples the machine-readable identification of the injection device is located at or close to the proximal end of the body or drive mechanism of the injection device. The detector arrangement may be located at or near a distal end of the body or drive mechanism of the injection device, which distal end is configured for fastening or mechanical coupling with the container part and/or with the medicament container located therein.

With some examples the detector arrangement provides a spatial range extender for the e.g. wireless communication link between the add-on device and the machine-readable identification as provided on or inside the injection device.

With some examples the machine-readable identification is electrically connected to the detector arrangement by at least one or numerous electrically conductive structures, such as by an electrically conductive wire. Hence, the electrical connection between the machine-readable identification and the detector arrangement may be implemented as a wire-based electrical coupling. Respective wires or electrically conductive structures may extend along a sidewall of the body and may thus extend along a longitudinal direction of the body, thus bridging the geometrical distance between the detector arrangement at or near the distal end of the body and the machine-readable identification at or near the proximal end of the body.

In some examples the container part is detachably connectable to the body in such a way that it is transferable between a connected configuration and a released configuration. In the connected configuration the container part is fastened, e.g. rigidly fastened to the body. In the released configuration the container part may be detached from the body. Here, it may be entirely detached, i.e. disconnected from the body such that the container part is completely separated from the body. In other examples and when the container part is in the released configuration it may remain connected to the body but may be in a configuration that allows to remove the medicament container and/or to insert a new medicament container into the container part. Here, the container part may be permanently connected to the body and may be movable relative to the body between the connected configuration and the released configuration. In some examples, the container part may be pivotally attached to the body. It may be pivoted with regard to a pivot axis between the connected configuration and the released configuration.

According to a further example the detector arrangement is configured to enter into a first detector state when the body is connected to the container part and is further configured to enter into a second detector state when the body is disconnected from the container part. The first detector state and the second detector state are electronically or electrically readable.

Hence, the status of the detector arrangement, i.e. the first detector state and the second detector state distinguish by an electrically measurable or electrically detectable feature of the detector arrangement.

With some examples one of detaching the container part from the body and attaching the container part to the body induces or triggers a switching of one of the first and the second detector state into the other one of the first and the second detector state.

First and second detector states of the detector arrangement may introduce respective electrically or electronically readable first and second identifier states of the machine-readable identification. Hence, the machine-readable identification may be or switch into a first identifier state when the detector arrangement is in the first detector state. The machine-readable identification may be or switch into a second identifier state when the detector arrangement switches into the second detector state.

Here, the first identifier state may reflect a first machine-readable state of the machine-readable identification and the second identifier state may reflect or constitute a second machine-readable state of the machine-readable identifier. The first and the second machine-readable states of the machine-readable identification distinguish from each other. The add-on device is particularly configured to wirelessly communicate with the machine-readable identification and to identify and/or recognize if the machine-readable identification is in the first machine-readable state or in the second machine-readable state.

According to a further example the machine-readable identification and the detector arrangement are electrically connected by an electrical circuit. This way, the first detector state and/or the second detector state is/are rather easily trackable or recognizable. Electric or electronic signals and/or electrically measurable properties of the detector arrangement can be transmitted via the electrical circuit to the machine-readable identification. This way, the detector state of the detector arrangement can be easily transmitted via the machine-readable identification to the add-on device when the add-on device is suitably attached and/or mechanically connected to the injection device.

According to a further example the electric circuit interconnecting the machine-readable identification and the detector arrangement comprises a first conductive wire and a second conductive wire. The first and the second conductive wires are connected to opposite ends of the detector arrangement. They are further connected to different terminals of the machine-readable identification.

By way of a first and a second conductive wire connected to e.g. first and second terminals of the machine-readable identification, a change of the detector state can be detected at the terminals of the machine readable identification. A change of the detector state is then immediately transferable into a respective change of input signals at the first and second terminals of the machine-readable identification, which in response to a respective change or modification of input signals changes its machine readable state. The respective change of the machine-readable state is directly trackable or detectable by the add-on device when attached to the injection device.

According to a further example the detector arrangement comprises an electromechanical switch on or in one of the body and the container part. The other one of the body and the container part comprises actuating element configured to engage the electromechanical switch when the container part is connected to the body. Furthermore, the actuating element is configured to disengage the electromechanical switch when the container part is disconnected from the body.

With some examples the electromechanical switch comprises a first switch terminal and a second switch terminal that are electrically insulated from each other and which, per default, are electrically disconnected from each other. Here, the actuating element may provide an electrically conductive structure, which serves as an electrical bridge between the first switch terminal and the second switch terminal when the container part is correctly connected to the body of the injection device. By detaching the container part from the body the electrical bridge as provided by the actuating element is subject to a movement and hence subject to a geometric displacement relative to the switch terminals, which motion or movement is accompanied by an electrical disconnection of the actuating element from at least one of the first and second switch terminals.

With other examples the actuating element is or comprises a mechanical component that interacts with an electrical bridge, which is an integral component of the electromechanical switch. With some examples the electromechanical switch comprises a movable part, e.g. in form of a contact spring, which is movable between an open state and a closed state. When in the closed state the contact spring provides electrical contact between first and second switch terminals and hence an electrical contact between first and second conductive wires of the electrical circuit. In an open state or open configuration the contact spring is disconnected from at least one of the first switch terminal and the second switch terminal and it is hence disconnected from at least one of the first and the second conductive wires.

A movement of the contact spring of the electromechanical switch may be induced or may be inducible or controllable by the actuating element. This way and upon interconnecting the body and the container part the actuating element may induce a movement of the contact spring from a closed state into an open state or vice versa from an open state into the closed state. Upon disconnecting the container part and the body the actuating element moves in the opposite direction thus inducing a closing or opening of the contact spring.

By way of the electromechanical switch electrically connected to the machine-readable identification a mechanical configuration of the injection device, i.e. the mechanical connection between the container part and the body, which is provided at the distal end of the body of the injection device, can be electrically detected at an opposite longitudinal end of the body of the injection device, where the machine-readable identification is typically located. This allows to implement a wireless near field or short range communication between the add-on device and the machine-readable identification with a wireless transmission range that can be shorter than the longitudinal distance between the machine-readable identification and the detector arrangement, which are typically located at opposite longitudinal ends of the body of the injector device.

According to a further example one of the body and the container part comprises an insert section. The other one of the body and the container part comprises a receptacle sized to receive the insert section. The detector arrangement is provided on an outside surface of the insert section or on an inside surface of the receptacle. Typically, when the detector arrangement is provided on an inside surface of the receptacle the actuating element is provided on an outside surface of the insert action. Vice versa, if the detector arrangement is provided on an outside surface of the insert section the actuating element is typically provided on an inside section of the receptacle.

With a further example the electromechanical switch is provided on one of the insert section and the receptacle. The actuating element is provided on the other one of the insert section and the receptacle.

When correctly assembled and connected the body and the container part are arranged in an at least partially interleaved or nested arrangement, in which the insert section overlaps with the receptacle. This way, a mechanically stable mutual interconnection between the container part and the body can be provided.

With a further example the electromechanical switch is provided on or in a fastening structure of the body. The actuating element is provided in or on a counter fastening structure of the container part, wherein the container part and the body are mutually fastenable to each other through a mechanical engagement of the fastening structure with the counter fastening structure. This way, the fastening structure and the complementary shaped counter fastening structure provide a twofold function. First of all, they are configured to provide a mechanical connection between the body and the container part of the injection device. Second, they provide an operation of the detector arrangement to modify the detector state of the detector arrangement in the course of assembly.

According to a further example one of the fastening structure and the counter fastening structure comprises a groove. The other one of the fastening structure and the counter fastening structure comprises a protrusion to engage the groove. With some examples the groove is implemented as a L-shaped groove or as a bayonet groove. It may be also provided in form of a threaded structure and may thus feature a helical shape.

With some examples the groove is provided on an inside surface of the receptacle. The protrusion is provided on an outside surface of the insert section. With other examples the groove may be provided on the outside surface of the insert section whereas the protrusion is provided on an inside surface of the sidewall of the receptacle.

With some examples the groove is provided as a threaded structure. The protrusion is provided as a complementary shaped counter threaded structure. This way, the fastening structure and the complementary shaped counter fastening structure enable a threaded engagement between the body and the container part.

According to a further example the body comprises a distal end and an oppositely located proximal end. The machine-readable identification is located nearer to the proximal end than the detector arrangement and/or the detector arrangement is located nearer to the distal end than the machine-readable identification.

Here, the electrical circuit between the machine-readable identification and the detector arrangement is operable to provide an electrical and hence geometric bridging between the machine-readable identification at or near a proximal end of the body and the detector arrangement at or near the distal end of the body of the injection device.

According to a further example the machine-readable identification comprises an electronic circuit. The electronic circuit comprises an antenna and an integrated circuit, which is operable to wirelessly communicate with the electronic module of the add-on device. With some examples the machine-readable identification comprises one of a RFID tag, NFC tag or UWB tag. The machine-readable identification may be implemented as a passive tag or as an active tag, the latter of which comprising an own energy source.

According to a further example the machine-readable identification comprises a memory configured to store at least one of a device information, a container information and a use-related data.

The use-related data may be indicative of a status of use of the injection device. The status of use may be determinable by the add-on device. The status of use may contain information such as: total number of doses set or injected, a total amount of medicament injected by the injection device, a type, a name and/or concentration of medicament injected by the injection device, a LOT number of a medicament container and other medicament-or medicament container-related information that may be useful for the actual or future use of the injection device.

The container information may be indicative of at least one of a type of a medicament contained in the medicament container a concentration of a pharmaceutical substance of the medicament contained in the container, a manufacturing date of the medicament, a use by date of the medicament or, a LOT number of the medicament or medicament container and/or environmental parameters, such as temperature, humidity or radiation intensity to which the medicament container has been exposed.

The device information may comprise information or data with regard to the type of the injection device. The device information may be specific about a type of an injection mechanism or drive mechanism of the injection device. The device information may thus characterize the dose setting and/or dose dispensing operability of the respective injection device. The device identification may further contain data or information about suitable medicaments or medicament containers to be exclusively used with the respective injection device.

According to a further example the integrated circuit comprises a first terminal and a second terminal. The first terminal is connected to one end of the detector arrangement and the second terminal is connected to another end of the detector arrangement. This way, the first and second terminals constitute different terminals of the machine-readable identification as mentioned above.

The first and the second terminal may be provided with machine-readable and hence electrically detectable signals from the detector arrangement when the body and the container part are mutually connected and attached and/or when body and the container part are disconnected.

According to a further example the integrated circuit comprises a general-purpose input/output (GPIO) terminal connected to one of the first terminal and the second terminal by or via a resistor. This way, a change of the detector state can be easily electronically detected at the GPIO terminal, which may be at a low or high voltage depending on the configuration of the detector arrangement, e.g. depending on the configuration of the electromechanical switch.

With a further example the general-purpose input/output terminal and the other one of the first terminal and the second terminal are connected via a capacitor, hence an input capacitor of the integrated circuit. With the first terminal and the second terminal of the integrated circuit being directly connected to the switch terminals of the electromechanical switch it may be required that the electronic module regularly conducts a request of the detector arrangement. Here, the electronic module may induce or trigger an actual status request of the detector arrangement by the machine-readable identification. For instance, the add-on device may be configured to conduct or to trigger such a checkup or request in regular time intervals, such as every 1-3 seconds. This way, the add-on device may be configured to regularly check the status of the detector arrangement.

With a GPIO terminal and with the resistor connected to one of the first terminal and the second terminal of the integrated circuit the resistor provides a pull up resistance for an improved or well defined signal detection. Here, the add-on device may be also configured to regularly detect the actual status of the detector arrangement, e.g. by regularly transmitting a respective request to the machine-readable identification. With the pull up resistance at the terminals of the integrated circuit a well-defined voltage level can be provided at the respective input terminals and/or at the GPIO terminal of the electronic circuit. This way, a rather precise and well-defined voltage measurement can be provided.

With the further example, wherein the GPIO terminal and the other one of the first terminal and the second terminal of the integrated circuit are connected by a capacitor a kind of an automated wake-up function can be implemented. Here, the capacitor may be operable to discharge when the electrical circuit interconnecting the detector arrangement and the machine-readable identification is opened, e.g. in the course of disconnecting the body and the container part. Discharge of the capacitor may induce an electronic wake-up of the integrated circuit, thereby automatically generating a respective wireless signal, which can be detected by the add-on device when attached to the injection device. Generation of a wake-up signal may also lead to a respective wake-up of the act on device. Insofar, that can be provided a rather outer numbers wake up function for the add-on device when attached to the injection device. Here, disconnection of the container part from the body or connection of the container part to the body may induce the generation of a respective wake up signal at the machine-readable identification, which in turn may induce a wake up of the add-on device.

This way, a regular supervision of the actual state of the detector arrangement may by the add-on device may become superfluous and respective electrical energy for conducting such regular checkups or requests can be saved.

With a further example the drive mechanism comprises at least one movable component, which is subject to a movement relative to the housing or medicament container during at least one of setting of the dose and injecting of the dose of the medicament. With some examples the movable component is one of a dose dial being rotatable relative to the housing for setting of a dose, a dial sleeve rotatable relative to the housing during setting of the those or dispensing of the dose, a drive sleeve or a drive member, which is subject to a rotational and/or longitudinal motion relative to the housing during at least one of setting of the dose and dispensing of the dose. With some examples the movable component is a clicker or clicking element operable to generate an audible sound recordable by the sensor of the add-on device. With further examples the movable component is a piston rod operably engaged with a piston of the medicament container for displacing the piston relative to the medicament container during injecting of the dose. With further examples the movable component is a single dose indicating member, whose movement and/or position relative to the housing or device body is indicative of the size of a dose currently set. With further examples the movable component is a last dose member, whose position relative to the housing of the injection device is directly indicative of the residual amount of medicament left in the medicament container or cartridge.

According to another aspect the present disclosure relates to an add-on device for attaching to an injection device as described above. The add-on device comprises a device body fastenable to a portion of the injection device. The add-on device further comprises at least one of a reader and a transceiver operable to read the machine-readable identification of the injection device and to generate a reading signal. The add-on device further comprises an electronic module comprising a module processor coupled to at least one of the reader and/or transceiver.

The module processor is operable to process the reading signal in order to determine if the container part of the injection device is connected to the body. With some examples the at least one of the reader and the transceiver is an integral part or a component of the electronic module of the add-on device.

Generally, the add-on device is configured to operate with an injection device as described above. Insofar, all features, effects and benefits as described above in connection with the injection device equally apply to the add-on device; and vice versa.

Specifically, the add-on device is operable to read or to read-out the machine-readable identification, which is electrically connected to the detector arrangement of the injection device. Via a read-out of the machine-readable identification the add-on device is capable to obtain or to read the detector state of the detector arrangement and is therefore enabled to distinguish between at least two different states of the detector arrangement indicating a mutual fastening of the container part and the body or a disconnection of the container part from the body.

With some examples the device body of the add-on device is detachably fastenable or connectable to a proximal end of the body of the injection device and/or to a proximal end of the injection mechanism or drive mechanism of the injection device. With some examples the device body comprises a receptacle sized to receive a dose dial or a dial extension of the injection device.

According to a further example the module processor is configured to sample or to request the reading signal from one of the reader and the transceiver according to a predefined time interval.

In the same way, the at least one of the reader and the transceiver of the add-on device is or are operable to sample or to request a connection-indicating signal from the machine-readable identification and/or from the detector arrangement of the injection device. With some examples the sample rate may be in the region of a few seconds. Hence, the add-on device may be configured to sample or to request a reading signal from the machine-readable identification and/or from the detector arrangement of the injection device via the machine-readable identification every 1-3 seconds. In this way, the add-on device is operable to constantly or regularly monitor the connection state of the injection device, which connection state is indicative of the mutual fastening between the body and the container part.

According to a further example the add-on device comprises a sensor operable to quantitatively determine at least one of a position and a movement of a movable component of the drive mechanism relative to at least one of the housing, the medicament container and the device body. The sensor is further operable to generate a respective sensor signal. The module processor of the add-on device is operable to determine or to calculate a size of the dose of the medicament, which is set or dispensed by the injection device, on the basis of the sensor.

According to a further example the at least one of the reader and the transceiver comprises a wireless near field transceiver operable to wirelessly communicate with the machine-readable identification of the injection device in order to obtain a state information of the detector arrangement and/or to obtain at least one of a device information and a container information stored in the machine-readable identification.

With some examples the at least one of the reader and the transceiver of the add-on device is implemented as a radiofrequency transceiver, e.g. as a RFID reader, NFC reader or UWB reader.

Since the machine-readable identification is electrically connected to the detector arrangement and since any change of the detector state unalterably reflects in a respective change of the configuration of the machine-readable identification the state or status of the detector arrangement can be read by the add-on device when the at least one of the reader and the transceiver wirelessly communicate with the machine-readable identification as provided on or inside the injection device.

According to a further example the at least one of the reader and the transceiver comprises a wireless local range transceiver. The wireless local range transceiver is operable to wirelessly communicate with an external electronic device to receive a state information of the detector arrangement and/or to receive at least one of a device information and a container information stored in the machine-readable identification from or via an external electronic device.

The local range transceiver may comprise a transmission range that is larger than the transmission range of the near field transceiver. The local range transceiver may comprise a radiofrequency transceiver. It may comprise a Bluetooth transceiver or a Bluetooth low energy (BLE) transceiver. It may also comprise a Wi-Fi transceiver or the like wireless transceiver allowing for a local range signal transmission with the external electronic device.

Typically, the external electronic device comprises one of a smart phone, a smartwatch and a tablet computer. With a communication link between the add-on device and the external electronic device it is also possible that the device information and/or container information as provided by the machine-readable identification is provided to the electronic module of the add-on device via the external electronic device. Here, the external electronic device may be equipped with a reader or transceiver operable to read-out the machine-readable identification of the injection device. In response to such a read-out of the machine-readable identification by the external electronic device the external electronic device may then be capable to extract the device information and/or the container information from the machine-readable identification and to transmit at least one or both of the device information and the container information to the add-on device via the local range transceiver of the add-on device. This way, the add-on device is indirectly provided with the information or data contained in or provided by the machine-readable identification of the injection device. With this example the add-on device may be void of a near field transceiver and does not necessarily have to be capable to obtain or to read the machine-readable identification by itself. Here, the external electronic device may operate as a relay, which reads or obtains the device information and/or the container information from the machine-readable identification and which is further operable to provide or to transmit the gathered information to the add-on device.

Of course, the local range transmission between the electronic module of the add-on device and the external electronic device requires some kind of an authentication procedure or pairing, such that unauthorized reading or transmission of device information and/or container information can be effectively controlled and/or prevented.

With some examples the module processor is further operable to determine or to calculate the size of the dose not only on the basis of the sensor signal but also on the basis of at least one of the container information and the device information as obtained from the machine-readable identification.

With the present add-on device, and by the above described acquisition of at least one of a device information and a container information a somewhat automated calibration of the dose size determination or dose size calculation can be provided. Hence, the add-on device itself is capable to derive and/or to process medicament container specific information and/or device specific information, e.g. in the course of assembly to the injection device. In response to obtain the container information and/or the device information the add-on device may be configured to conduct a calibration routine. This way the measurable sensor signals as obtained by the sensor of the add-on device can be translated or re-calculated into correct dose size information.

According to a further example the electronic module further comprises a module memory connected to the module processor. The module memory and/or the module processor is or are operable to store at least one of the device information and the container information as stored information and/or to store a dosing history, wherein the dosing history includes a number of dose sizes dispensed or injected by the injection device and determined or calculated by the module processor.

According to a further example the module processor is operable to determine or to calculate a residual amount of the medicament contained in the medicament container on the basis of the dosing history and on the basis of a connection status between the body and the container part. With some examples a dose size or dose information obtained from the sensor of the add-on device is only and exclusively taking into account for calculating of a residual amount of the medicament as long as the add-on device determines or confirms that the container part is and remains connected to the body of the injection device. Accordingly, the add-on device, in particular the electronic module and/or the module processor thereof is operable to determine the connection status between the container part and the body of the injection device in a way as described above, namely by reading the machine-readable identification, which is electrically connected to the detector arrangement. A size of a dose currently set or dispensed is then only used for calculation of a residual amount of medicament contained in the medicament container if the add-on device determines or confirms that the container part and the body of the injection device remain mutually fixed and/or connected.

For this the add-on device is operable to regularly check for the mutual connection between the container part and the body, e.g. by sampling or requesting a reading signal from one of the reader and the transceiver, which, when the add-on device is correctly assembled or attached to the injection device, can is operable to wirelessly communicate with the machine-readable identification, thereby obtaining respective information about the detector state of the detector arrangement of the injection device.

With some examples the add-on device may be configured to interrupt or to discard a calculation of a residual amount of the medicament contained in the medicament container if a mechanical disconnection the container part and the body should be detected. Disconnecting the container part from the body may lead to a change of the state of the detector arrangement, which status change is electronically readable via the machine-readable identification. Detection of a status change during the ongoing and regular processing of dosing history monitoring and/or residual amount calculation is a safety feature to avoid calculation of a wrong residual amount of the medicament left in the medicament container in the event that a user should detach the container part from the body prematurely, i.e. before the content of the medicament container has been completely dispensed or used up.

With further examples and when the machine-readable identification is provided with at least one of a container information and a device information the detection of the container part from the body may be tolerable, especially when the dosing history as obtained, determined or calculated by the add-on device is written or stored in a memory of the machine-readable identification. Reattaching the container part to the body may then enable a reading of the respective machine-readable identification and to resume a residual amount calculation of the respective container part or medicament container.

According to another aspect the present disclosure further relates to an injection system comprising an injection device as described above and further comprising an add-on device as described above. Insofar, any features, effects and benefits as described above in connection with any of the injection device and the add-on device equally apply to the injection system.

The injection system may additionally comprise at least one of an external electronic device operable to communicate, e.g. to wirelessly communicate with the add-on device. The external electronic device may be implemented as a smartphone, as a smartwatch, and a tablet computer or any other digital electronic device that is capable to communicate with the add-on device.

According to another aspect the present disclosure relates to a method of monitoring operation of an injection device, which injection device is configured for injecting of a dose of a medicament. The method comprises the steps of attaching of an add-on device to a portion of the injection device, wherein the injection device comprises a machine-readable identification on or inside one of a body and a container part of a housing of the injection device. The machine-readable identification is electrically connected to detector arrangement. The detector arrangement is provided on or inside one of the body and the container part. The detector arrangement is operable to detect, e.g. to electrically detect, a mechanical connection between the body and the container part.

The method further comprises the step of reading of the machine-readable identification by at least one of a reader and a transceiver of the add-on device and generating a reading signal being indicative of a mechanical connection between the body and the container part. The method further comprises the step of determining if the container part is connected to the body by processing of the reading signal. Typically, the method of monitoring operation of the injection device is to be executed and/or conducted by an injection device as described above and/or by making use of an add-on device as described above. Insofar, all features, effects and benefits as described above in connection with any one of the injection device and the add-on device equally apply to the method of monitoring operation of the injection device as described herein.

According to a further example the method comprises the steps of quantitatively determining at least one of a position and a movement of the movable component of a drive mechanism of the injection device by a sensor of the add-on device when the add-on device is attached to the injection device. The sensor provides and generates a respective sensor signal being indicative of a degree of movement and/or being indicative of a position information by way of which a position of the movable component relative to any one of the housing of the injection device and the device body of the add-on device can be detected or determined.

By quantitatively detecting or measuring a degree of movement of the movable component of the drive mechanism there can be derived dose size information being indicative of a size of a dose currently set or dispensed by the injection device. Quantitatively measuring the dose size allows to monitor and to log any dosing event of the injection device and to record a respective dosing history.

Accordingly, and with a further example the method comprises the step of monitoring a dosing history with the add-on device, wherein the dosing history includes a number of dose sizes dispensed, e.g. sequentially dispensed or injected by the injection device and determined or calculated by the add-on device. Storing of the dosing history may include a point of time at which a dose has been set and/or dispensed as well as the amount of medicament set or dispensed with each dispensing or injection action. The dosing history may be stored locally in the add-on device. It may be synchronized e.g. through wireless data transmission with the external electronic device and may be shared with other entities or persons, such as healthcare providers.

With some examples a dosing history may be stored in a memory of a machine-readable identification of the injection device. Insofar, the recorded dosing history can be stored on-site and directly on the injection device or component thereof that has been used to set and to dispense respective doses of the medicament.

According to a further example the method comprises the step of determining or calculating a residual amount of the medicament contained in a medicament container on the basis of the dosing history and on the basis of a connection status between the body and the container part. In this way and particularly by taking into account the connection status between the body and the container part any unintended use or misuse of the injection device can be retrieved and checked by the detector arrangement and the machine-readable identification of the injection device. Specifically, a premature detaching or replacement of a medicament container can be rather autonomously detected and can be used to discard or to interrupt an ongoing dosing history calculation and/or a residual amount of medicament calculation or determination. In this way it can be prevented, that incorrect dosing information is generated and/or used to calculate the residual amount of the medicament inside a medicament container.

According to a further aspect the present disclosure also relates to a computer program, e.g. a computer program product comprising computer readable instructions, which when executed by a module processor of an add-on device as described above cause the module processor to conduct the steps of the method as described above.

Typically, the computer program is executable by an add-on device as described above. It is further operable to conduct a method of monitoring operation of the injection device with the add-on device as described above. Insofar, the computer program and its computer readable instructions are operable to cause a processor of the add-on device to conduct a method of monitoring or checking the injection device as described above. The computer readable instructions may be executable by a processor of an add-on device as described above.

Additionally, or alternatively and when an external electronic device of an injection system as described above is configured to read-out the machine-readable identification of the injection device the computer program and its computer readable instructions may be at least in part executable by a processor of the external electronic device.

The computer program is to be executed by a processor of an add-on device as described above in order to monitor and/or record operation of the injection device when the add-on device is attached thereto. Insofar, all features, effects and benefits as described above in connection with the add-on device and/or described in connection with the method of configuring the add-on device as well as any effects, features and benefits as described above in connection with the injection system equally apply to the computer program; and vice versa. The present disclosure further discloses and proposes a computer program including computer-executable instructions for performing the method according to the disclosed method/device/system in one or more of the examples enclosed herein when the program is executed on a processor, computer or computer network. Specifically, the computer program may be stored on a computer-readable data carrier. Thus, specifically, one, more than one or even all of the method steps as indicated above may be performed by using a computer or a computer network, typically by using a computer program.

The present disclosure further discloses and proposes a computer program product having program code means, in order to perform the method according to the disclosed method/system in one or more of the embodiments enclosed herein when the program is executed on a computer or computer network. Specifically, the program code means may be stored on a computer-readable data carrier.

Further, the present disclosure discloses and proposes a data carrier having a data structure stored thereon, which, after loading into a processor, computer or computer network, such as into a working memory or main memory of the processor, computer or computer network, may execute the method according to one or more of the examples disclosed herein.

The present disclosure further proposes and discloses a computer program product with program code means stored on a machine-readable carrier, in order to perform the method or parts thereof according to one or more of the examples disclosed herein, when the program is executed on a processor, computer or computer network. As used herein, a computer program product refers to the program as a tradable product. The product may generally exist in an arbitrary format, such as in a paper format, or on a computer-readable data carrier. Specifically, the computer program product may be distributed over a data network.

With a further example the injection device comprises a dose dial and a trigger, e.g. provided at a proximal longitudinal end of the injection device. The injection device may be implemented as a pen-type injector. The dose dial and/or the trigger may be provided at a proximal longitudinal end of the pen-type injector. Here, the machine-readable identification may be provided in or on the dose dial and/or the trigger. The add-on device may be configured for releasable or detachable fastening to the proximal end of the injection device.

The add-on device, in particular its device body may comprise a receptacle to fit onto the dose dial and/or trigger. By implementing or arranging the machine-readable identification in or on the trigger or dose dial a geometric distance between the machine-readable identification and a reader or transceiver of the add-on device for readout of the machine-readable identification can be reduced to a minimum thus allowing to implement the transceiver or reader of the add-on device as a wireless near field transceiver operable to read a respective near field communication tag of the machine-readable identification of the device. This way, readout of the machine-readable identifier can only take place when the add-on device is correctly mounted to the proximal end of the injection device.

According to a further example the injection system comprises an external electronic device, e.g. implemented as a smartphone, a smart watch or as a tablet computer. The external electronic device may be operable to conduct at least one or several steps of the method of monitoring operation of the injection device.

Generally, the scope of the present disclosure is defined by the content of the claims. The disclosure is not limited to specific embodiments or examples but comprises any combination of elements of different embodiments or examples. Insofar, the present disclosure covers any combination of claims and any technically feasible combination of the features disclosed in connection with different examples or embodiments.

In the present context the term ‘distal’ or ‘distal end’ relates to an end of the injection device that faces towards an injection site of a person or of an animal. The term ‘proximal’ or ‘proximal end’ relates to an opposite end of the injection device, which is furthest away from an injection site of a person or of an animal.

The terms “drug” or “medicament” are used synonymously herein and describe a pharmaceutical formulation containing one or more active pharmaceutical ingredients or pharmaceutically acceptable salts or solvates thereof, and optionally a pharmaceutically acceptable carrier. An active pharmaceutical ingredient (“API”), in the broadest terms, is a chemical structure that has a biological effect on humans or animals. In pharmacology, a drug or medicament is used in the treatment, cure, prevention, or diagnosis of disease or used to otherwise enhance physical or mental well-being. A drug or medicament may be used for a limited duration, or on a regular basis for chronic disorders.

As described below, a drug or medicament can include at least one API, or combinations thereof, in various types of formulations, for the treatment of one or more diseases. Examples of API may include small molecules having a molecular weight of 500 Da or less; polypeptides, peptides and proteins (e.g., hormones, growth factors, antibodies, antibody fragments, and enzymes); carbohydrates and polysaccharides; and nucleic acids, double or single stranded DNA (including naked and cDNA), RNA, antisense nucleic acids such as antisense DNA and RNA, small interfering RNA (siRNA), ribozymes, genes, and oligonucleotides. Nucleic acids may be incorporated into molecular delivery systems such as vectors, plasmids, or liposomes. Mixtures of one or more drugs are also contemplated.

The drug or medicament may be contained in a primary package or “drug container” adapted for use with a drug delivery device. The drug container may be, e.g., a cartridge, syringe, reservoir, or other solid or flexible vessel configured to provide a suitable chamber for storage (e.g., short-or long-term storage) of one or more drugs. For example, in some instances, the chamber may be designed to store a drug for at least one day (e.g., 1 to at least 30 days). In some instances, the chamber may be designed to store a drug for about 1 month to about 2 years. Storage may occur at room temperature (e.g., about 20° C.), or refrigerated temperatures (e.g., from about −4° C. to about 4° C.). In some instances, the drug container may be or may include a dual-chamber cartridge configured to store two or more components of the pharmaceutical formulation to-be-administered (e.g., an API and a diluent, or two different drugs) separately, one in each chamber. In such instances, the two chambers of the dual-chamber cartridge may be configured to allow mixing between the two or more components prior to and/or during dispensing into the human or animal body. For example, the two chambers may be configured such that they are in fluid communication with each other (e.g., by way of a conduit between the two chambers) and allow mixing of the two components when desired by a user prior to dispensing. Alternatively or in addition, the two chambers may be configured to allow mixing as the components are being dispensed into the human or animal body.

The drugs or medicaments contained in the drug delivery devices as described herein can be used for the treatment and/or prophylaxis of many different types of medical disorders. Examples of disorders include, e.g., diabetes mellitus or complications associated with diabetes mellitus such as diabetic retinopathy, thromboembolism disorders such as deep vein or pulmonary thromboembolism. Further examples of disorders are acute coronary syndrome (ACS), angina, myocardial infarction, cancer, macular degeneration, inflammation, hay fever, atherosclerosis and/or rheumatoid arthritis. Examples of APIs and drugs are those as described in handbooks such as Rote Liste 2014, for example, without limitation, main groups 12 (anti-diabetic drugs) or 86 (oncology drugs), and Merck Index, 15th edition.

Examples of APIs for the treatment and/or prophylaxis of type 1 or type 2 diabetes mellitus or complications associated with type 1 or type 2 diabetes mellitus include an insulin, e.g., human insulin, or a human insulin analogue or derivative, a glucagon-like peptide (GLP-1), GLP-1 analogues or GLP-1 receptor agonists, or an analogue or derivative thereof, a dipeptidyl peptidase-4 (DPP4) inhibitor, or a pharmaceutically acceptable salt or solvate thereof, or any mixture thereof. As used herein, the terms “analogue” and “derivative” refers to a polypeptide which has a molecular structure which formally can be derived from the structure of a naturally occurring peptide, for example that of human insulin, by deleting and/or exchanging at least one amino acid residue occurring in the naturally occurring peptide and/or by adding at least one amino acid residue. The added and/or exchanged amino acid residue can either be codable amino acid residues or other naturally occurring residues or purely synthetic amino acid residues. Insulin analogues are also referred to as “insulin receptor ligands”. In particular, the term “derivative” refers to a polypeptide which has a molecular structure which formally can be derived from the structure of a naturally occurring peptide, for example that of human insulin, in which one or more organic substituent (e.g. a fatty acid) is bound to one or more of the amino acids. Optionally, one or more amino acids occurring in the naturally occurring peptide may have been deleted and/or replaced by other amino acids, including non-codeable amino acids, or amino acids, including non-codeable, have been added to the naturally occurring peptide.

Examples of insulin analogues are Gly(A21), Arg(B31), Arg(B32) human insulin (insulin glargine); Lys(B3), Glu(B29) human insulin (insulin glulisine); Lys(B28), Pro(B29) human insulin (insulin lispro); Asp(B28) human insulin (insulin aspart); human insulin, wherein proline in position B28 is replaced by Asp, Lys, Leu, Val or Ala and wherein in position B29 Lys may be replaced by Pro; Ala(B26) human insulin; Des(B28-B30) human insulin; Des(B27) human insulin and Des(B30) human insulin.

Examples of insulin derivatives are, for example, B29-N-myristoyl-des(B30) human insulin, Lys(B29) (N-tetradecanoyl)-des(B30) human insulin (insulin detemir, Levemir®); B29-N-palmitoyl-des(B30) human insulin; B29-N-myristoyl human insulin; B29-N-palmitoyl human insulin; B28-N-myristoyl LysB28ProB29 human insulin; B28-N-palmitoyl-LysB28ProB29 human insulin; B30-N-myristoyl-ThrB29LysB30 human insulin; B30-N-palmitoyl-ThrB29LysB30 human insulin; B29-N-(N-palmitoyl-gamma-glutamyl)-des(B30) human insulin, B29-N-omega-carboxypentadecanoyl-gamma-L-glutamyl-des(B30) human insulin (insulin degludec, Tresiba®); B29-N-(N-lithocholyl-gamma-glutamyl)-des(B30) human insulin; B29-N-(ω-carboxyheptadecanoyl)-des(B30) human insulin and B29-N-(ω-carboxyheptadecanoyl) human insulin.

Examples of GLP-1, GLP-1 analogues and GLP-1 receptor agonists are, for example, Lixisenatide (Lyxumia®), Exenatide (Exendin-4, Byetta®, Bydureon®, a 39 amino acid peptide which is produced by the salivary glands of the Gila monster), Liraglutide (Victoza®), Semaglutide, Taspoglutide, Albiglutide (Syncria®), Dulaglutide (Trulicity®), rExendin-4, CJC-1134-PC, PB-1023, TTP-054, Langlenatide / HM-11260C (Efpeglenatide), HM-15211, CM-3, GLP-1 Eligen, ORMD-0901, NN-9423, NN-9709, NN-9924, NN-9926, NN-9927, Nodexen, Viador-GLP-1, CVX-096, ZYOG-1, ZYD-1, GSK-2374697, DA-3091, MAR-701, MAR709, ZP-2929, ZP-3022, ZP-DI-70, TT-401 (Pegapamodtide), BHM-034. MOD-6030, CAM-2036, DA-15864, ARI-2651, ARI-2255, Tirzepatide (LY3298176), Bamadutide (SAR425899), Exenatide-XTEN and Glucagon-Xten.

An example of an oligonucleotide is, for example: mipomersen sodium (Kynamro®), a cholesterol-reducing antisense therapeutic for the treatment of familial hypercholesterolemia or RG012 for the treatment of Alport syndrom. Examples of DPP4 inhibitors are Linagliptin, Vildagliptin, Sitagliptin, Denagliptin, Saxagliptin, Berberine. Examples of hormones include hypophysis hormones or hypothalamus hormones or regulatory active peptides and their antagonists, such as Gonadotropine (Follitropin, Lutropin, Choriongonadotropin, Menotropin), Somatropine (Somatropin), Desmopressin, Terlipressin, Gonadorelin, Triptorelin, Leuprorelin, Buserelin, Nafarelin, and Goserelin.

Examples of polysaccharides include a glucosaminoglycane, a hyaluronic acid, a heparin, a low molecular weight heparin or an ultra-low molecular weight heparin or a derivative thereof, or a sulphated polysaccharide, e.g. a poly-sulphated form of the above-mentioned polysaccharides, and/or a pharmaceutically acceptable salt thereof. An example of a pharmaceutically acceptable salt of a poly-sulphated low molecular weight heparin is enoxaparin sodium. An example of a hyaluronic acid derivative is Hylan G-F 20 (Synvisc®), a sodium hyaluronate.

The term “antibody”, as used herein, refers to an immunoglobulin molecule or an antigen-binding portion thereof. Examples of antigen-binding portions of immunoglobulin molecules include F(ab) and F(ab′)2 fragments, which retain the ability to bind antigen. The antibody can be polyclonal, monoclonal, recombinant, chimeric, de-immunized or humanized, fully human, non-human, (e.g., murine), or single chain antibody. In some embodiments, the antibody has effector function and can fix complement. In some embodiments, the antibody has reduced or no ability to bind an Fc receptor. For example, the antibody can be an isotype or subtype, an antibody fragment or mutant, which does not support binding to an Fc receptor, e.g., it has a mutagenized or deleted Fc receptor binding region. The term antibody also includes an antigen-binding molecule based on tetravalent bispecific tandem immunoglobulins (TBTI) and/or a dual variable region antibody-like binding protein having cross-over binding region orientation (CODV).

The terms “fragment” or “antibody fragment” refer to a polypeptide derived from an antibody polypeptide molecule (e.g., an antibody heavy and/or light chain polypeptide) that does not comprise a full-length antibody polypeptide, but that still comprises at least a portion of a full-length antibody polypeptide that is capable of binding to an antigen. Antibody fragments can comprise a cleaved portion of a full length antibody polypeptide, although the term is not limited to such cleaved fragments. Antibody fragments that are useful in the present invention include, for example, Fab fragments, F(ab′)2 fragments, scFv (single-chain Fv) fragments, linear antibodies, monospecific or multispecific antibody fragments such as bispecific, trispecific, tetraspecific and multispecific antibodies (e.g., diabodies, triabodies, tetrabodies), monovalent or multivalent antibody fragments such as bivalent, trivalent, tetravalent and multivalent antibodies, minibodies, chelating recombinant antibodies, tribodies or bibodies, intrabodies, nanobodies, small modular immunopharmaceuticals (SMIP), binding-domain immunoglobulin fusion proteins, camelized antibodies, and VHH containing antibodies. Additional examples of antigen-binding antibody fragments are known in the art.

The terms “Complementarity-determining region” or “CDR” refer to short polypeptide sequences within the variable region of both heavy and light chain polypeptides that are primarily responsible for mediating specific antigen recognition. The term “framework region” refers to amino acid sequences within the variable region of both heavy and light chain polypeptides that are not CDR sequences, and are primarily responsible for maintaining correct positioning of the CDR sequences to permit antigen binding. Although the framework regions themselves typically do not directly participate in antigen binding, as is known in the art, certain residues within the framework regions of certain antibodies can directly participate in antigen binding or can affect the ability of one or more amino acids in CDRs to interact with antigen. Examples of antibodies are anti PCSK-9 mAb (e.g., Alirocumab), anti IL-6 mAb (e.g., Sarilumab), and anti IL-4 mAb (e.g., Dupilumab).

Pharmaceutically acceptable salts of any API described herein are also contemplated for use in a drug or medicament in a drug delivery device. Pharmaceutically acceptable salts are for example acid addition salts and basic salts.

Those of skill in the art will understand that modifications (additions and/or removals) of various components of the APIs, formulations, apparatuses, methods, systems and embodiments described herein may be made without departing from the full scope and spirit of the present invention, which encompass such modifications and any and all equivalents thereof.

An example drug delivery device may involve a needle-based injection system as described in Table 1 of section 5.2 of ISO 11608-1:2014(E). As described in ISO 11608-1:2014(E), needle-based injection systems may be broadly distinguished into multi-dose container systems and single-dose (with partial or full evacuation) container systems. The container may be a replaceable container or an integrated non-replaceable container.

As further described in ISO 11608-1:2014(E), a multi-dose container system may involve a needle-based injection device with a replaceable container. In such a system, each container holds multiple doses, the size of which may be fixed or variable (pre-set by the user). Another multi-dose container system may involve a needle-based injection device with an integrated non-replaceable container. In such a system, each container holds multiple doses, the size of which may be fixed or variable (pre-set by the user).

As further described in ISO 11608-1:2014(E), a single-dose container system may involve a needle-based injection device with a replaceable container. In one example for such a system, each container holds a single dose, whereby the entire deliverable volume is expelled (full evacuation). In a further example, each container holds a single dose, whereby a portion of the deliverable volume is expelled (partial evacuation). As also described in ISO 11608-1:2014(E), a single-dose container system may involve a needle-based injection device with an integrated non-replaceable container. In one example for such a system, each container holds a single dose, whereby the entire deliverable volume is expelled (full evacuation). In a further example, each container holds a single dose, whereby a portion of the deliverable volume is expelled (partial evacuation).

### BRIEF DESCRIPTION OF THE DRAWINGS

In the following, numerous examples of a data logging device for monitoring use of an injection device as well as a respective injection device will be described in greater detail by making reference to the drawings, in which:

FIG. 1 schematically illustrates an example of an injection device,

FIG. 2 shows the process of assembling an add-on device to a proximal end of the injection device,

FIG. 3 shows an injection device in a longitudinal schematic cross-section,

FIG. 4 shows a configuration of an injection system including the injection device, the add-on device and at least one external electronic device,

FIG. 5 shows a pairing between the external electronic device and the add-on device,

FIG. 6 shows a visual illustration on a display of an external electronic device to assist a user in setting up the injection device,

FIG. 7 shows a configuration of the external electronic device indicating an end of dose configuration of the medicament container,

FIG. 8 shows the display of the external electronic device prompting the user to replace the medicament container,

FIG. 9 illustrates the display of the external electronic device providing statistic data of recent dose setting and injecting procedures,

FIG. 10 shows a display configuration of the external electronic device after replacement of a medicament container,

FIG. 11 shows a configuration of the add-on device before attempting to the proximal end of the injection device

FIG. 12 shows a visual feedback as provided on the external electronic device in response to a read-out of information from the electronic container identifier,

FIG. 13 shows an example of a container part is detached from the body of the injection device,

FIG. 14 separately illustrates the distal end of the body configured for connection with the proximal end of the container part,

FIG. 15 shows an example of the injection device and the add-on device before assembling the add-on device to the injection device,

FIG. 16 shows the add-on device assembled to the injection device,

FIG. 17 shows an example of an electromechanical switch of a detector arrangement,

FIG. 18 shows another example of an electromechanical switch of a detector arrangement,

FIG. 19 shows a further example of an electromechanical switch of a detector arrangement,

FIG. 20 shows a block diagram of the add-on device, the injection device and the external electronic device,

FIG. 21 shows a block diagram of the electronic circuit of the machine-readable identification,

FIG. 22 shows an example of the electronic circuit on a substrate,

FIG. 23 shows a flowchart of a method of configuring the add-on device.

### DETAILED DESCRIPTION

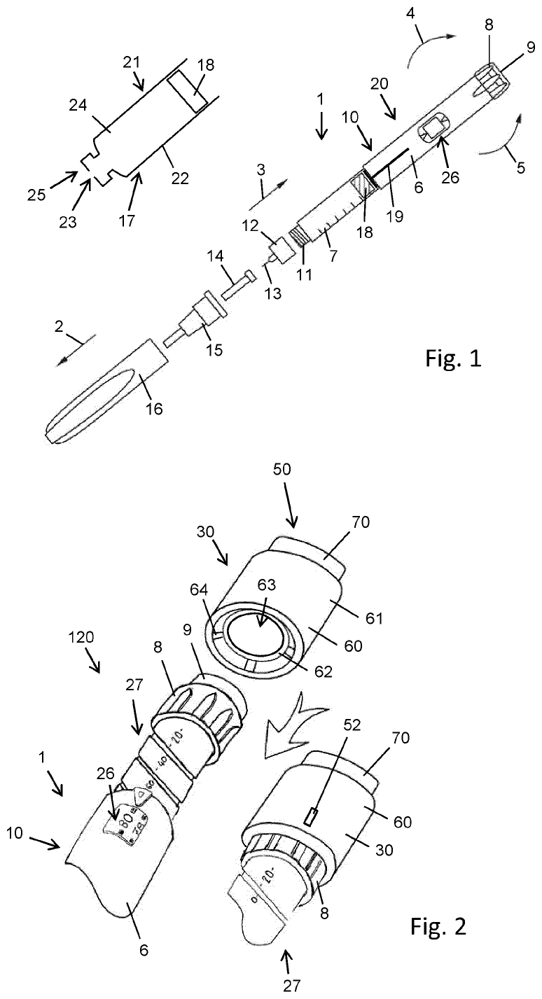

FIG. 1 shows an example of a drug delivery device, which is implemented as a handheld injection device 1. The injection device 1 may comprise or may be implemented as a pen-type injector. It may be implemented as a disposable injection device or as a reusable injection device. With some examples the injection device 1 is implemented as an autoinjector. The injection device 1 is of elongated shape. It may extend along a longitudinal direction. Towards a longitudinal distal direction 2 the drug delivery device 1 comprises a dispensing end for dispensing or injecting the medicament 24. Towards the proximal direction 3 the injection device 1 comprises at least one of a dose member 8 and a trigger 9, by way of which a dose of equal or individual or different size can be set and dispensed, respectively.

The injection device 1 comprises a housing 10. The housing 10 may comprise numerous housing components, such as a body 6 and a container part 7, e.g. implemented as a cartridge holder 7. The body 6 may be sized and configured to accommodate a drive mechanism 20. The container part 7 is sized and configured to accommodate a medicament container 21, e.g. implemented as a cartridge containing the liquid medicament 24. The medicament container 21 comprises a tubular-shaped barrel 22 sealed towards the distal end by a seal 23. The seal 23 may comprise a pierceable septum fixed to an outlet 25 of the medicament container 21. Towards a proximal end the interior of the barrel 22 is sealed by a piston 18 or stopper, which is slidably disposed inside the barrel 22.

By advancing the piston 18 towards the distal direction 2 a dose of the medicament 24 can be expelled from the medicament container 21. In use the medicament container 21 is arranged inside the container part 7. The drive mechanism 20 of the injection device 1 comprises a piston rod 19, which is displaceable in distal direction 2 for advancing the piston 18 towards the outlet 25 of the medicament container 21. Details of the drive mechanism are not further illustrated and described here. With some examples, the drive mechanism 20 may be implemented as an all-mechanical drive mechanism, where a user has to provide an entirety of a dispensing force required to move the piston rod 19 and hence the piston 18 in distal direction 2. With other examples, the drive mechanism comprises a mechanical energy storage configured to provide at least a portion of the dispensing force. Examples of drive mechanisms can be found e.g. in WO2004/078241 A1, WO 2014/033197 A1 or WO 2014/033195 A1 the entirety of which are herein incorporated by reference.

With some examples, as e.g. described or shown in FIG. 2 the injection device 1 and hence the drive mechanism 20 may comprise a dial extension 27, which projects and moves in proximal direction 3 from a proximal end of the body 6 when or during setting a dose and which returns into its initial distal end position during a dose injection procedure. For this, a user may use a thumb 114 of his hand 110 to exert a distally directed pressure onto the trigger 9 thereby urging the dial extension 27 in distal direction 2 during and/or for a dose injection procedure.

For setting or dialing of a dose a user may twist or rotate the dose dial 8, e.g. in a dose incrementing direction 4, hence in a clockwise sense as seen from the proximal end. For correcting a dose previously set the user may also rotate the dose dial 8 in an opposite dose decrementing direction 5. The size of the dose is typically illustrated in a window 26 provided in or on the body 6 of the injection device 1. Prior to inject a dose of the medicament 24 the distal end of the container part 7 or cartridge holder has to be connected with a needle assembly 12. For this, the distal end of the cartridge holder 7 comprises a connector 11, e.g. in form of a threaded interface to engage with a complementary shaped threaded counter interface of the needle assembly 12.

The needle assembly 12 is detachably or releasably fixable to the container part 7. It comprises a double-tipped injection needle 13. A proximal end of the injection needle (not shown) is configured to enter into a through opening at the distal end face of the connector 11 or container part 7 so as to pierce or to penetrate the seal 23 of the medicament container 21. The distal end of the injection needle 13 is typically covered by a detachable inner needle cap 14. The entirety of the needle assembly 12 may be covered by a detachable outer needle cap 15.

The container part 7 and hence a portion of the housing 10 is to be received in a protective cap 16, which is detachably connectable to the cartridge holder 7 or body 6.

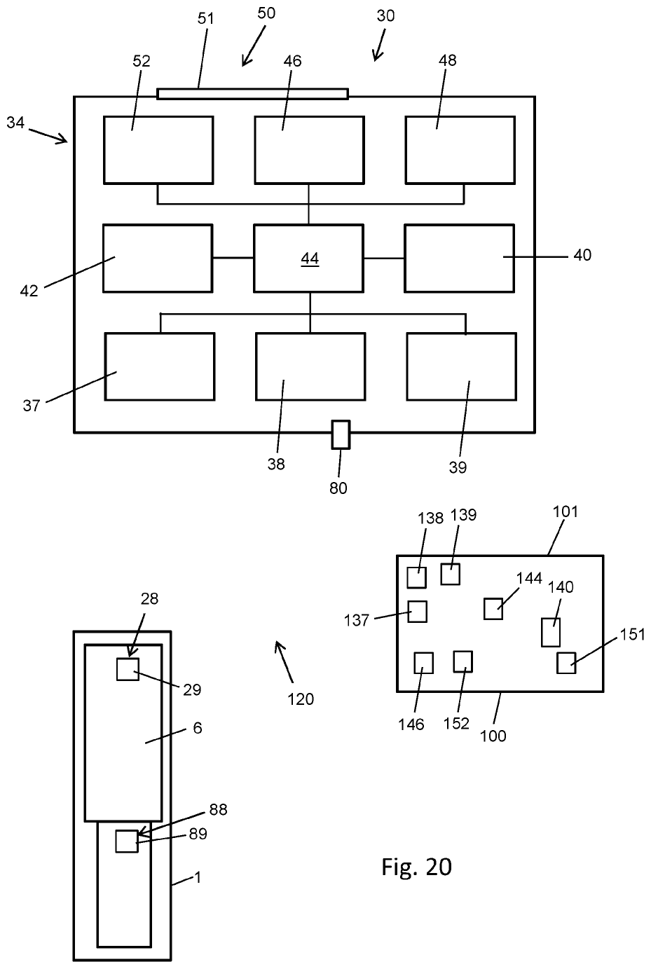

In FIGS. 2 and 4 there is shown an example of an add-on device 30 configured for fastening to the proximal end of the injection device 1. The add-on device 30 comprises a sensor 48 or sensor assembly, which is operable to detect, to recognize, to characterize and/or to measure an operation of the injection device 1. Typically, the sensor 48 of the add-on device 30 and as indicated in the block diagram of FIG. 20 is capable or operable to quantitatively determine or to measure at least one of a position and a movement of a movable component 80, 81, 82, 83 of the drive mechanism 20 of the injection device 1. The movable component 80, 81, 82, 83 is movable relative to at least one of the body 6 and the medicament container 21 during or for setting and/or injecting of a dose of the medicament 24.

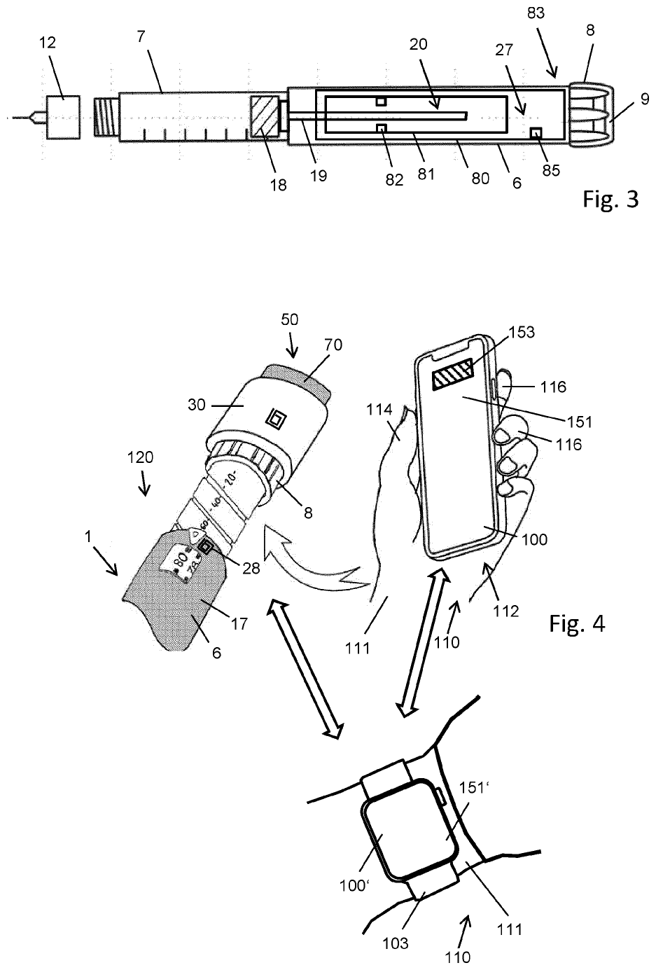

This movement or position is quantitatively measurable by the sensor 48. Typically, the sensor 48 is implemented as an electronic sensor. It is capable to detect or to measure a degree of longitudinal translation and/or rotation of the at least one movable component 80, 81, 82, 83 relative to the housing 10 or relative to the device body 60 when the add-on device 30 is attached to the injection device 1. Examples of movable components 80 are schematically illustrated in the cross-section of the injection device 1 according to FIG. 3 The sensor 48 of the add-on device 30 is capable or operable to generate a sensor signal or a sequence of sensor signals, which due to the coupling or connection to the module processor 44 can be processed by the module processor 44 in order to derive or to calculate a size of a dose currently set or dispensed.

With some examples the movable component 80 is a dose setting member or a dose setting sleeve. With further examples the movable component 81 is implemented as a drive member or drive sleeve 81 of the drive mechanism 20. With further examples the movable component is implemented as a volume indicator 82, e.g. as a single dose indicating member, whose position or configuration relative to the body 6 is directly indicative of the size of a dose currently set. With further examples the movable component 82, hence the volume indicator is a last dose indicating member, the position or configuration of which relative to the body 6 is indicative of a remaining amount of medicament left in the cartridge or medicament container 21.

With some examples the movable component 83 is or comprises an encoding, such as a digital encoding, which is movable relative to the sensor 48 such that the sensor is capable to count a number of discrete code portions of the encoding so as to gather respective quantitative movement information of the movable component 80. Generally, there may be provided different types of spatial encodings, such as an optical or visual encoding, an electrical or electrostatic encoding a magnetic encoding or mechanical encoding. Depending on the type of an encoding provided on or inside the movable component 80, 81, 82, 83 the sensor 48 is correspondingly configured. Hence, the sensor 48 may comprise at least one of an optical sensor, an electrical or electrostatic sensor and a magnetic sensor or mechanical sensor, such as a micromechanical switch, each of which being operable to quantitatively measure a relative position or relative movement of the movable component 80, 81, 82, 83.

With some examples the movable component 80, 81, 82, 83 is subject to a longitudinal and/or rotational movement relative to the body 6, relative to the medicament container 21 and/or relative to the device body 60 during at least one of setting of the dose and dispensing of the dose. Typically, the encoding as provided on or with the movable component 80, 81, 82, 83 is encoded along the direction of movement of the movable component relative to the sensor 48 so as to enable a respective quantitative measurement of the respective dose setting or dose dispensing movement.

As further indicated in FIG. 3 the drive mechanism 20 may be optionally equipped with a locking mechanism 85, which is operable by a locking controller of the add-on device 30 when the add-on device 30 is correctly assembled to the injection device 1. The locking mechanism 85 is operable to block or to lock the drive mechanism 20 so as to prevent unauthorized or unintended setting of a dose and/or dispensing or injecting of the dose. The locking mechanism 85 is controllable by the locking controller 86 of the add-on device 30. The locking mechanism 85 may be implemented electromechanically and may be electrically or electronically controlled by the locking controller 86 of the add-on device 30. This way, the add-on device 30 may block an unauthorized or unintended use of the injection device 1.

The add-on devices 30 as illustrated in the various FIGS. 2-14 is detachably connectable to the dose dial 8. It comprises a device body 60 with a tubular-shaped sidewall 61. Towards the distal end the sidewall 61 confines a receptacle 63, which is sized to receive the dose dial 8 and the trigger 9 of the injection device 1. For this, the inside of the sidewall 60 may comprise one or numerous fastening ribs 64, which are configured to provide a slip free fastening of the add-on device 30 to the dose dial 8.

The device body 60 comprises the receptacle 63 at a distal end section of the add-on device 30. The receptacle 63 is open towards the distal direction 2. It is sized and configured to fit onto the proximal end of the injection device 1. An inside surface of the sidewall 61 confining the receptacle 63 comprises numerous fastening ribs 64, e.g. of an elastic or elastically deformable material, such as an elastomeric material.

The fastening ribs 64 provide a friction fit with the dose dial 8 when the device body 60 is assembled to the dose dial 8 of the injection device 1. The receptacle 63 may be confined in longitudinal direction by a flange portion 62 protruding radially inwardly from the tubular-shaped sidewall 61 of the receptacle 63. The flange portion 62 is located proximally offset from the insert opening of the receptacle 63. The flange portion 62 may be of annular shape and may be configured to axially abut on a stepped down section at the proximal end of the dose dial 8. Hence, the trigger 9, which comprises a reduced diameter compared to the dose dial 8 may protrude in proximal direction 3 through the flange portion 62 while the flange portion 62 may rest or abut against the proximal end face of the dose dial 8 when the add-on device 30 is suitably fastened to the dose dial 8.

The add-on device 30 further comprises a movable part 70 protruding in proximal direction from the device body 60. The movable part 70 comprises or forms an auxiliary trigger or trigger button, which is configured to mechanically engage with the trigger 9 of the injection device 1 when the add-on device 30 is correctly assembled to the injection device 1.

Typically, the add-on device 30, in particular, the device body 60 is frictionally engageable with the dose dial 8. In this way, a user may apply a dose setting torque onto the dose dial via the device body 60. Instead of rotating the dose dial 8 for setting of a dose the user may simply rotate the device body 60 relative to the body 6 of the injection device 1. This way, the dial extension 27 may become subject to a dose incrementing dialing or rotating motion. A respective size of a dose currently set will then be displayed in the window 26 of the body 6 of the injection device 1 as illustrated in FIG. 2.

For dispensing of a dose the user has to depress the movable part 70, which may then be subject to a distally directed motion relative to the device body 60. The movable part 70, which may be in direct or indirect mechanical engagement with the trigger 9, may then apply a respective dispensing force onto the trigger 9 thereby initiating a dose dispensing action of the injection device 1.

The add-on device 30 as schematically illustrated in FIG. 20 comprises an electronic module 34. The electronic module 34 comprises a printed circuit board 36. The electronic module 34 comprises a module processor 44, an electronic and hence digital module memory 40 and a clock 42. Furthermore, the electronic module 34 comprises a power source 46 and the sensor 48. The electronic module 34 also comprises a signal generator 52 coupled to the processor 44 and/or coupled to the power source 46. The electronic module 34 further comprises at least one of a reader 37 and a wireless transceiver 38, 39. The add-on device 30 may also comprise a user perceptible device identification 50.

With some examples the device identification 50 comprises or includes the signal generator 52 coupled to the module processor 44 and operable or reconfigurable by the module processor 44. With some examples the device identification 50 may be indicative of the injection device 1 to which the add-on device 30 is actually connected or coupled to.

With some examples the signal generator 52 is operable to generate at least one of a visual, an acoustic and a haptic signal or device identification and is hence operable to produce or to generate a visual identifier, an acoustic identifier or a haptic identifier or a respective alert signal. With some examples the add-on device 30 comprises numerous signal generators 52 of equal or different type, e.g. optical, acoustic or haptic type.

With the example of FIG. 2 the signal generator 52 may comprise a speaker operable to generate an acoustic signal, wherein the acoustic signal may represent or constitute an alert signal e.g. in form of an acoustic code or sequence being characteristic for a specific configuration or setting of the injection device 1 or add-on device 30.

FIG. 4 is illustrative of an injection system 120 comprising at least the add-on device 30 and the injection device 1. Optionally, the injection system 120 comprises one or several external electronic devices 100, 101′. The external electronic device 100 is implemented as a smart phone comprising a housing 101 and being equipped with a device display 151 to visually illustrate various types of information, such as various illustrations 153 or notifications 154 to a user. The display 151 may be implemented as a touch sensitive display. The device display 151 may be operable to provide or to emulate a device signal generator 152. The device display 151 is operable to provide at least one of an illustration 153 and a notification 154 to a user.

The further optional external electronic device 100′is implemented as a smartwatch comprising a respective housing and a display 151′and further comprising a wristband 103 for fixing the external electronic device 100′to the hand 110 or wrist 111 of a user. Like the external electronic device 100 also the external electronic device 100′is operable to provide at least one of an illustration 153 and a notification 154 to the user. Generally, the external electronic device 100, 100′may be equipped with a separate device signal generator 152, such as a speaker or a vibration module by way of which a user perceivable signal can be generated so as to provide an alert signal to a user of the respective device The electronic device 100 is typically implemented as a handheld electronic device. It can be held in a hand 110 of a user or may be worn on the wrist 111 of a user. With the example as illustrated in FIG. 4 the electronic device 100 is held in a palm 112 of the user. The device display 151 is implemented as a touch sensitive display and can be operated by a thumb 114 and/or by various fingers 116 of the user's hand 110.

The electronic module 34 of the add-on device 30 as schematically illustrated in FIG. 20 comprises at least one of a reader 37 and a transceiver 38, 39 operable for wireless communication with at least one of a machine-readable identification 28, 88 of the injection device 1 or with a complementary or correspondingly implemented device reader 137 and/or device transceiver 138, 139 of the external electronic device 100, 100′.

With typical examples the transceivers 38, 138 are implemented as radiofrequency transceivers of near field type. The transceivers 38, 138 may be implemented as wireless short range transceivers or near field transceivers. They may be implemented as a so-called NFC transceivers allowing for wireless communication within a limited spatial range of a few centimeters or decimeters. The transceivers 38, 138 may be implemented as active and/or passive NFC tags or readers. Typically, the transceiver 38 is implemented as a passive NFC tag and the transceiver 138 is implemented as an active NFC tag or NFC reader.

The transceivers 39, 139 may be implemented as wireless local range transceivers, such as RFID, Bluetooth, Bluetooth low energy (BLE) or UWB transceivers. Also here, the transceiver 39 may be implemented as a passive communication tag whereas the transceiver 139 may be implemented as an active transceiver, hence as a reader. Typically, the transmission range of the transceivers 39, 139 is larger than the transmission range of the transceivers 38, 138. The transmission range of the transceivers 39, 139 may be in a range of several meters or decameters.

The optional reader 37, 137 may be implemented as an optical reader or optical information gathering unit, e.g. comprising an imaging system so as to capture or to record a visual or optical machine-readable identification 28, 88 as provided on or inside the injection device 1.

The external electronic device 100, 100′typically comprises a device processor 144 coupled to an electronic device memory 140. The electronic device 100 further comprises a device power source 146. Also, the electronic device 100 comprises a device display 151, typically implemented as a touch sensitive display. The device display 151 may be operable to provide at least one of an illustration 153 and a notification 154 to a user.

The device transceiver 138 is operable to communicate with the transceiver 38 of the add-on device. The transceiver 139 is configured to communicate with the transceiver 39 of the add-on device 30. The reader 137 may be operable to read a visual identifier as provided on the injection device 1. With some examples the device reader 137 comprises an imaging system. It may comprise a camera objective and a spatially resolving detector in order to read or to capture a visual code, e.g. provided on an outside surface of the injection device 1 or as provided on an outside surface of the medicament container 21.

The external device 100 is configured to set up a communication link, e.g. a wireless communication link with the add-on device 30. For this, the local range transceiver 39 of the add-on device 30 may communicate wirelessly with the local range device transceiver 139 of the add-on device 100. Once a communication link has been established between the electronic device 100 and the add-on device 30 the electronic device 100 may be operable to visibly illustrate information or data of the injection device or medicament container 21 on the device display 151

The injection device 1 is provided with a machine-readable identification 28, 88 as schematically illustrated in FIG. 20. The machine-readable identification 28 comprises an electronic identifier 29. The machine-readable identification 88 comprises an electronic identifier 89. The electronic identifier 28 may comprise a passive wireless communication tag as schematically illustrated in FIG. 12.

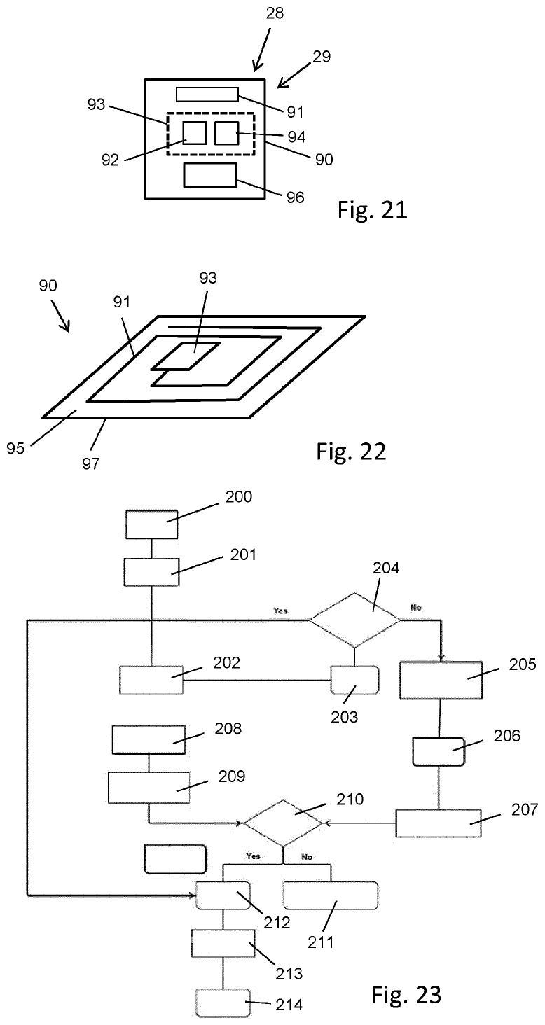

As illustrated in FIG. 21 the electronic identifier 29 may comprise an electronic circuit 90 featuring a wireless communication antenna 91 and an integrated circuit 93. The integrated circuit 93 comprises an electronic and hence digital memory 92 to and a processor 94 connected to the memory 92. Of course, the integrated circuit 93 and hence the processor 94 is connected to the antenna 91. The electronic identifier 29 may optionally comprise a power source 96 in order to provide electrical power or energy for the processor 94. The electronic circuit 90 may be provided on a planar substrate 95. The substrate 95 may be pliable or foldable. It may comprise a flexible structure or foil that allows for an easy mounting, fastening e.g. adhesive fastening, of the substrate 94 and hence of the entire electronic identifier 29 to at least one of the tubular shaped body 6, the container part 7 or the medicament container 21.

The electronic circuit 90 may be a printed electronic circuit, which is printed on the substrate 95. Here, the substrate 95 may comprise or constitute an e.g. flexible printed circuit board. The substrate 95 may be further provided with an adhesive layer 97 that allows for an easy and straightforward adhesive attachment of the machine-readable identification 28, 88 to a predefined portion on or inside the injection device 1. The electronic identifier 89 may comprise an identical or similar structure as the electronic identifier 29 as shown in FIGS. 12 and 13.

With some examples the machine-readable identification 28, 88 is implemented in a label 17 located on one of the body 6, the container part 7 or the medicament container 21. The label 17 comprises or contains the machine-readable identification 28, 88. With some examples the label 17 comprises a passive radiofrequency tag. With further examples the label 17 comprises machine-readable identifications 28, 88 implemented as a visual or optical code that can be captured by a reader 37 or device reader 137, which is implemented as or comprises an imaging system.

At least one of the reader 37, the device reader 137 or at least one of the transceivers 38, 39 or device transceivers 138, 139 is or are operable to read the machine-readable identification 28, 88 thereby obtaining at least one of a device information and a container information e.g. stored in the memory 92 of the electronic circuit 90.

Typically, the machine-readable identification 28 is provided or located on or inside the body 6 of the housing 10 of the injection device 1. The further machine-readable identification 88 is provided on or inside the container part 7. It may be provided on or inside the medicament container 21. With some examples the machine-readable identification 28 may be provided in or on the piston 18 of the medicament container 21.

The machine-readable identification 28, 88 may be implemented as a passive RF communication tag, such as a NFC tag. The information, e.g. the device information and/or the container information stored in at least one of the machine-readable identification 28, 88 is readable by the electronic module 34 and hence by any one of the transceivers 38, 39 of the add-on device 30 and/or by any of the transceivers 138, 139 of the external electronic device 100, 100′.

With some examples and especially when the injection device 1 is implemented as a disposable injection device, which does not support replacement of the medicament container 21 it is sufficient when the injection device 1 comprises only one machine-readable identification 28. It may be of particular advantage when the machine-readable identification 28 is provided at or near a proximal end 68 of the body 6. It may be provided on or inside the dose dial 8 or the trigger 9 so as to be within the transmission range of the transceiver 38, 39 when the add-on device 30 is attached to the proximal end of the injection device 1.

With some examples the distal end 67 of the body 6 is detachably connectable to a proximal end of the container part 7. Then, the injection device 1 is implemented as a reusable device that allows a replacement of the container part 7 and/or a replacement of the medicament container 21 inside the container part 7. With reusable injection devices 1 it may be of particular benefit when both, the body 6 and the container part 7 are provided with a separate machine-readable identification 28, 88. As illustrated in FIG. 20, the container part 7 is provided with the machine-readable identification 88 whereas the body 6 is provided with the machine-readable identification 28.

With some examples the transceiver 38 may be located inside the receptacle 63 of the device body 60 so as to minimize a spatial distance to the machine-readable identification 28 as provided at or near the proximal end 68 of the body 6. By making use of near field communication technology for the machine-readable a notification 28, 88 and the respective transceivers 38, 138 it can be ensured, that the machine-readable identification and the device information and/or container information stored therein can only be acquired or obtained when the add-on device 30 and/or the external electronic device 100, 100′is within the near field transmission range.

As further illustrated in FIG. 15, the receptacle 63 of the device body comprises an alignment feature 65 in form of a longitudinal recess or groove on the inside of the sidewall of the receptacle 63. Correspondingly, the proximal end of the dose dial 8 comprises a complementary shaped counter alignment feature 66, e.g. in form of a protrusion sized to fit into the recess or groove of the alignment feature 65. In this way, there can be provided a rotation inhibiting fastening for the add-on device 30 when attached to the housing 10 or dose dial 8 of the injection device 1.

When the alignment feature 65 is in mechanical engagement with the counter alignment feature 66 as shown in FIG. 16 there is provided an anti-rotation lock for the mutual fastening of the add-on device 30 to the injection device 1. In this way there can be guaranteed, that the identification 28 and hence the electronic identifier 29 always correctly aligns with the associated wireless transceiver 38 of the add-on device 30. There is hence provided a unique and well-defined mounting position for mounting the add-on device 30 to the injection device 1. When reaching the mounting position or mounting configuration as predefined by alignment feature 65 engaging the counter alignment feature 66 it is also guaranteed, that the machine-readable identification 28 and the transceiver 38 are at a minimum distance to each other. This allows for a somewhat undisturbed signal transmission between the identification 28 or electronic identifier 29 and the transceiver 38.

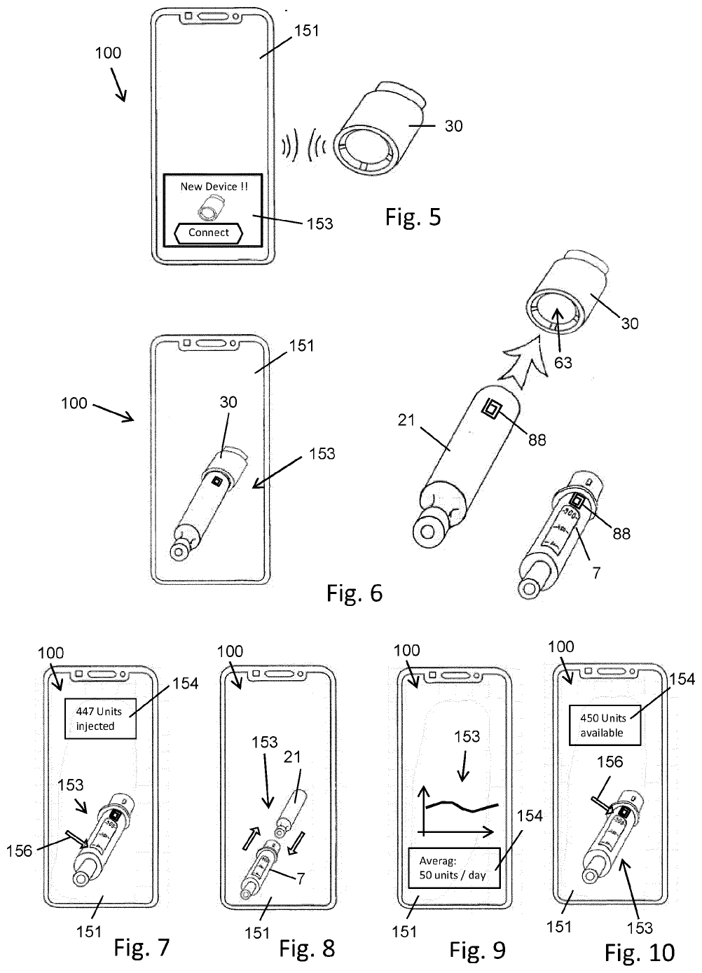

Generally, the external electronic device 100 may permanently or regularly synchronize with the add-on device e.g. by local range communication interfaces as provided by the transceivers 39, 139. Insofar, any information gathered or read by the add-on device 30 can be instantly transmitted to and further processed, e.g. visualized, by the external electronic device 100, 100′. The sequence of FIGS. 5-12 illustrates one of a plurality of examples of how to use the add-on device 30 in connection with the injection device one. In the configuration of FIG. 5 a user of the external electronic device is informed of a pairing between the external electronic device 100 and the add-on device 30. Such a pairing may be obtained through establishing of a wireless communication link between the transceiver 39 and the device transceiver 139. Once the external electronic device 100 detect the presence and/or correct pairing with the add-on device it is operable to provide a respective visual illustration one hundred and 53 on the device display one hundred and 51.

In a subsequence step the external electronic device 100 may be operable to assist or to guide a user to bring a medicament container 21 enclosed vicinity to the add-on device 30 so as to enable a reading of the machine-readable identification 88 as provided on the medicament container 21. This way and when the medicament container 21 provided with the machine-readable identification 88 is e.g. inserted in the receptacle 63 of the add-on device 30 the add-on device 30 is operable to read-out the respective container information. Alternatively and as illustrated on the right-hand side of FIG. 6 it is also conceivable, that the container part 7 of the injection device one is provided with a respective machine-readable identification.

Read-out of the machine-readable identification may reveal in the configuration as illustrated in FIG. 7, that a comparatively large amount of medicament units have already been injected and hence dispensed from the medicament container 21. An empty medicament container may be provided in form of a visual illustration one hundred and 53 on the device display one hundred and 50 one. Here, the almost empty configuration of the medicament container 21 may be accompanied by a virtual indicator 156 concurrently provided on the device display one hundred and 51 together with an illustration of the medicament container 20 one or container part 7.

Thereafter and as illustrated in FIG. 8 the add-on device 100 may prompt the user to replace the medicament container 21 with regard to the container part 7. Hence, the empty medicament container 21 should be removed from the container part 7 and 8 new medicament container 21 should be inserted into the container part 7.

Optionally and as illustrated in FIG. 9 the external electronic device may provide statistic data or statistic illustrations one hundred and 53 e.g. of a recent use of the injection device one. After having inserted the new medicament container 21 inside the container part 7 and after having e.g. conducted a repeated reading procedure as described above in connection with FIG. 6 the external electronic device 100 may provide a visual illustration one hundred and 54 of a new and hence sealed medicament container 20 one or container part 7 together with a respective indicator 156. In addition, the may be provided a notification one hundred and 54 on the device display one hundred and 51 indicating to the user the number of available medicament units provided in the medicament container 21 and/or provided in the container part 7.

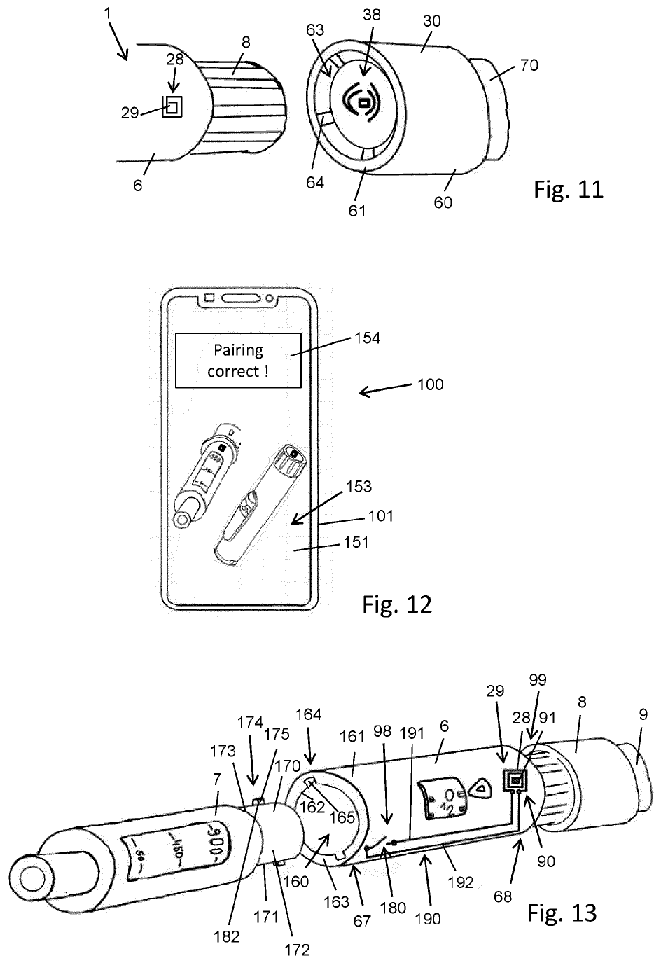

FIG. 11 illustrates a scenario before the add-on device 30 is attached to the proximal end of the injection device one. Here, the transceiver 38 of the add-on device 30 is located inside the receptacle 63. Upon mounting of the add-on device 30 to the proximal end of the injection device one, hence to the dose dial 8 a spatial distance between the transceiver 38 and the electronic device identification 29 can be reduced to a minimum thus allowing to read the configuration of the electronic device identification 29 and/or to read-out information stored in the electronic device identification 29.

In case that the container information as obtained from the electronic container identifier 89 matches with the device information as obtained from the electronic device identifier 29 the external electronic device 100 may be configured to indicate to a user the correct pairing of the body 6 and the container part 7 as illustrated in FIG. 12. Both, the body 6 and the container part 7 can be individually identified by way of their respective electronic body identifier 29 and electronic container identifier 89. The matching of the medicament container 21 and/or the container part 7 with the body 6 may be visually illustrated on the display 151 of the external electronic device 100, e.g. in form of an illustration 153 of the recognized or identified components and/or by a respective notification 154 indicating to a user that the individually recognized or identified components of the injection device 1 mutually match.

Generally, the external electronic device 100 may permanently or regularly synchronize with the add-on device e.g. by local range communication interfaces as provided by the transceivers 39, 139. Insofar, any information gathered or read by the add-on device 30 can be instantly transmitted to and further process, e.g. visualized by the external electronic device 100, 100′.

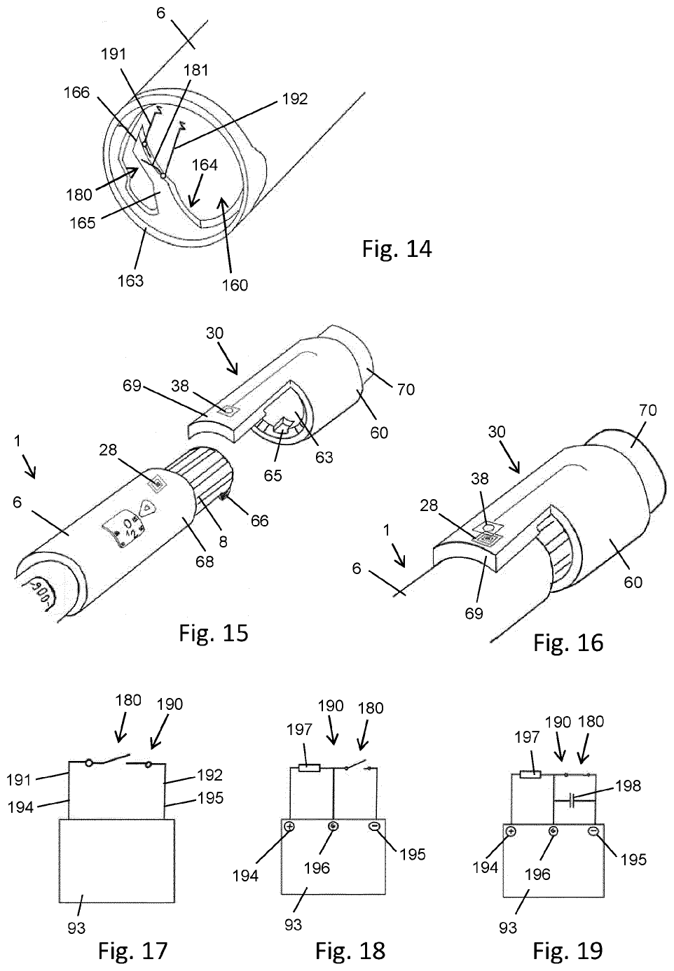

With the example of FIGS. 13 and 14 the injection device 1 comprises a detector arrangement 98 on or inside the body 6, which is operable to detect a mechanical connection between the body 6 and the container part 7. The machine-readable identification 28 is electrically connected to the detector arrangement 98. Due to the electrical connection between the detector arrangement 98 and the machine-readable identification 28 the machine-readable identification becomes indicative of the mechanical connection between the body 6 and the container part 7. This way and by reading the machine-readable identification 28, e.g. by the add-on device 38 a status of the detector arrangement 98 can be detected and hence determined.

The mechanical connecting status between the container part 7 and the body 6 is hence electronically readable by the machine-readable identification 28. Typically, and as illustrated in FIG. 13 the machine-readable identification 28 may be implemented as a RFID tag 99, as a NFC tag or as a UWB tag.

Depending on the mechanical connection between the container part 7 and the body 6 the detector arrangement 98 is switchable in one of a first detector state and a second detector state. If the body 6 is connected to the container part 7 the detector arrangement 98 is in the first detector state. When the body 6 is disconnected from the container 7 the detector arrangement 98 is in the second detector state. Both, the first and the second detector states are electronically readable through the machine-readable identification 28.

As illustrated in greater detail in FIGS. 13 and 14 the machine-readable identification 28 and the detector arrangement 98 are electrically connected by an electrical circuit 190. The electrical circuit 190 comprises a first conductive wire 191 and a second conductive wire 192. Moreover, the detector arrangement 98 comprises an electromechanical switch 180. For instance, and when the switch 180 is closed the detector arrangement 98 is in the first detector state. When the switch 180 is opened the detector arrangement 98 is in the second detector state.

With other examples the electromechanical switch 180 is closed when the detector arrangement is in the second detector state. The electromechanical switch 180 is opened when the detector arrangement 98 is in the second detector state.

With the example as illustrated in FIG. 13 the electromechanical switch 180 is operable to interrupt the electrical circuit 190. Hence, when the switch 180 is opened the electrical connection between the detector arrangement 98 and the machine-readable identification 28 is interrupted. By closing the electromechanical switch 180 the electrical connection between the detector arrangement 98 and the machine-readable identification 28 is established.

The electrical circuit 190 may be directly electrically connected or integrated with or in the electronic circuit 90 of the machine-readable identification 28 and/or its electronic identifier 29. With some examples and by opening of the switch 180 e.g. a connection between the antenna 91 and the integrated circuit 93 of the electronic circuit 90 may be interrupted and the machine-readable identification 28 may be disabled to wirelessly communicate with the add-on device 30.

It may be only and exclusively upon closing of the switch 180, that the electronic circuit 90 is closed and that the electronic identifier 29 and hence the machine-readable identification 28 is enabled to communicate with the add-on device 30.

With the example of FIGS. 13 and 14 the distal end 67 of the body 6 is provided with a receptacle 165 to receive a complementary shaped insert section 170 as provided at a proximal end of the container part 7. The body 6 comprises a sidewall 161 confining the receptacle 160. The sidewall 161 is provided with a distal end face 163 facing in distal direction 2. The end face 163 is configured to engage, i.e. to longitudinally abut with a stepped portion 173 provided on the outside surface of the sidewall 171 of the container part 7.

Here, the insert section 170 protrudes in proximal direction 3 from the stepped down portion 173. The insert section 170 comprises a smaller diameter compared to the tubular portion of the sidewall 171 extending distally from the stepped down portion 173.

On the inside surface 162 of the receptacle 160there is provided a fastening structure 164 complementary shaped to a counter fastening structure 174 on the outside surface 172 of the sidewall 171 of the container part 7 and hence on the insert section 170. The fastening structure 164 comprises a groove 165 configured to engage with a complementary shaped protrusion 175 of the counter fastening structure 174.

The groove 165 may comprise one of a L-shaped groove and a bayonet groove as illustrated in greater detail in FIG. 14. The groove 165 may be delimited in tangential or circumferential direction by a stop 166 providing a well-defined end stop for the mutual connection between the fastening structure 164 and the counter fastening structure 174. Alternative to the illustrated example it is also conceivable to provide an inner thread on the inside surface 162 and an outer thread on the outside surface one 172.

The electromechanical switch 180 may comprise a movable contact spring 181, which in the open configuration as illustrated in FIG. 14 extends at least partially into the groove 164. The contact spring 181 is permanently connected to the second conductive wire 192. Due to the inherent spring force the contact spring 181 tends to disengage from the first conductive wire 191. It is only and exclusively upon establishing a mechanical connection between the container part 7 and the body 6 that an actuating element 182 as provided on the outside surface 172 of the insert section 170 engages the contact spring 181 thus bringing the contact spring 181 in electrical connection with the first conductive wire 191, thereby closing the electrical circuit 190. The actuating element 182 may coincide with the protrusion 175 as provided on the outside surface 172 of the insert section 170. By attaching and hence fastening the insert section 172 to the receptacle 160 the protrusion 175 slides along the groove 165 until it reaches the stop 166. When in this particular stop position the protrusion 175 is engaged with the contact spring 181 and establishes a respective electrical connection between the first conductive wire 191 and the second conductive wire 192.

Accordingly and when reaching a mutual fastening configuration between the body 6 and the container part 7 the electrical circuit 190 is closed.

In FIG. 17 there is illustrated one example of an implementation of the electrical circuit 190. Here, the switch 180 may be directly connected to first and second input terminals 194, 195 of the integrated circuit 93 of the machine-readable identification 28. With the example of FIG. 17 opening of the switch 180 may disconnect the terminals 194, 195, thus disabling a wireless communication function of the integrated circuit 93. Here, and by opening of the switch 180 the integrated circuit 93 may be disconnected from the antenna 91. It may be only upon closing of the switch 180 that there is provided an electrical connection between the antenna 91 and the integrated circuit 93 that enables a wireless communication function of the machine-readable identification 28.

With this example it is required or intended that the add-on device 30, when attached to the injection device 1, regularly submits or requests status information from the machine-readable identification 28. Here, the add-on device 30 may be configured to transmit or to send a respective request to the machine-readable identification. As long as the container part 7 is not connected to the body part 6 and as long as the switch 180 is opened the machine-readable identification 28 is unable to respond to the add-on device's 30 requests.

With the further example of FIG. 18 the integrated circuit 93 is provided with a GPIO terminal 196 in addition to first and second input terminals 194, 195. Here, the first terminal 194 and the GPIO terminal 196 are mutually connected by a resistor 197. The switch 180 is provided between the other input terminal 195 and the GPIO terminal. This way there is provided a pull-up resistor for an improved signal detection. Hence, when the switch 180 is closed there is provided a well-defined voltage at the GPIO terminal 196, which is easily detectable by the integrated circuit 93.

With the example of FIG. 18 it is also required or intended that the add-on device 30, when attached to the injection device 1, regularly transmits or requests status information from the machine-readable identification 28 being indicative of a configuration or state of the detector arrangement 98.

With the further example according to FIG. 19 the configuration of FIG. 18 is slightly modified. Here, the GPIO terminal 196 and the second input terminal 195 are mutually connected via a capacitor 198. When the switch 188 is closed the capacitor 198 will be charged.

By opening of the switch 180, e.g. in the course of disconnecting the container part 7 from the body 6 the capacitor 198 a discharge of the capacitor can be detected by the integrated circuit 93 and/or the discharging capacitor temporally provides electric power to the integrated circuit to e.g. to submit a state information to the add-on device 30. Hence, the capacitor 198 may be configured to at least temporally provide electric energy to the integrated circuit 93 when the switch 180 is open.

The example of FIG. 19 may also provide a kind of an automated wake-up function for the integrated circuit 93, namely when closing or opening of the switch 180. With the example of FIG. 19 it might be of particular benefit to keep the switch 180 closed when the container part 7 is disconnected from the body 6 and to open the switch 180 when attaching the container part 7 to the body 6.

It is also conceivable that the switch 180 is kept open when the container part is disconnected from the body 6 and that the switch 180 closes when the container part 7 is attached and connected to the body 6. In either way and by way of the capacitor 198 the integrated circuit 93 may be operable to obtain electrical power to wirelessly indicate a change of the state of the switch arrangement 98, which is readable and/or detectable by the add-on device 30 when attached to the injection device 1.

Implementation of the detector arrangement 98 on or inside the body of the injection device 1 is beneficial in order to enable an electronic reading or electronic detection if the container part 7 is correctly assembled or attached to the body 6. Here, it is sufficient when the injection device is void of any active electric or electronic components. The detector arrangement 98 may be implemented exclusively by the electromechanical switch 180 connected to the integrated circuit 93 of the electronic device identifier 28, which may be implemented as a passive radiofrequency tag.

The electrical connection between the detector arrangement 98 and the machine-readable identification 28 allows to detect and/or to record a mechanical connection of the body 6 with the container part 7, wherein a proximal end of the container part 7 is connectable or is connected with a distal end 67 of the body 6. The machine-readable identification 28 may be provided at or near the proximal end 68 of the body 6 and may provide a near field communication with the add-on device 30 within a transmission range that is shorter than the longitudinal distance between the detector arrangement 98 and the machine-readable identification 28.

For recording a dosing history and/or for calculating a residual amount of medicament left in the medicament container 21 the add-on device 30 is operable to constantly and/or to regularly check the status of the detector arrangement 98 so as to assert that the connection between the container part 7 and the body 6 is maintained during recording of the dosing history and/or during calculating of the residual amount of medicament left in the medicament container 21.

In the flowchart of FIG. 23 a typical scenario of use of the injection system 120 is schematically illustrated. In an initial step 200 the add-on device 30 is assembled to the injection device 1. In the course of an initial assembly the add-on device 30 may be suitably calibrated. In step 201 a dose history recording is be started.

In an optional step 202 status information being indicative of the configuration of the injection device 1 and/or or being indicative of a configuration or of particular characteristics of the medicament container 21 can be written or stored in any of the memories 92 of the electronic device identifier 28 and/or of the electronic container identifier 89.

In steps 202 and/or 203 the presence of the electronic device identifier 28 and hence of the machine-readable identification 28 is repeatedly and hence somewhat permanently monitored by requesting a respective response from the machine-readable identification 28. In step 203 the add-on device 30 may be and remain in a respective standby mode, during which standby mode the respective presence requests with the electronic device identifier 29 and hence with the machine-readable identification 28 is regularly executed.

In step 204 it is checked if the communication link between the add-on device 30 and the machine-readable identifier 28 has been lost. If the communication between the machine-readable identification 28 and the add-on device 30 should be interrupted the method proceeds with step 212. Then, and in a subsequent step 213 the electronic module 34 generates an alert, which may be indicated to the user via the signal generator 52. For instance, a blinking light implemented by the identifier 55, e.g. integrated into the movable part 70 of the add-on device 30, may provide a visual alert signal, e.g. in form of a blinking light.

In a subsequent step 214 the add-on device 30 may communicate with the external electronic device 100 to stop calculating a residual amount of the medicament of the respective medicament container. Here, a recorded dosing history and/or residual amount calculation may be discarded in order to avoid that a patient or health care provider uses such information, which due to a premature exchange of the medicament container 21 might have become invalid.

As long as in step 204 there is confirmed that the machine-readable identification 28 is present and/or indicates that the container part 7 is and remains connected to the body 6 the procedure continues with step 205. Here, a user may set a dose to be injected by the injection device. Conducting of a dose setting may wake-up the sensor 48 of the electronic module 34 in step 206.

In the following step 207 and after or during dispensing of a dose of the medicament the respective dose dispensing event is recorded. The recorded dose injection in step 207 is then subject to a plausibility check in step 210. Here, the recorded or detected dose dispensing information is compared or confirmed with status parameters of the injection device 1 and/or with status parameters of the medicament container 21.

In step 208 it is detected if a user disconnects the container part 7 from the body 6. A reconnection of the container part 7 to the body 6 may be also recorded and/or detected in step 208. In a subsequent step 209 a respective modified status of the injection device 1 may be recorded and/or stored in the memory 92 of the electronic device identifier 29 and hence in the machine-readable identification 28.

The status information as provided by the machine-readable identification 28 may be always compared with a recorded dosing event of step 207. This comparison is conducted in step 210. Typically, such a plausibility check shall be conducted before, during or after each dose injection recording. This enables the add-on device 30 to check the status of the connection between the container part 7 and the body 6 at the latest time and simplifies, e.g. reduces the power consumption of the add-on device 30. Here, there may be required only one wireless reading request between the machine-readable identification 28 and the add-on device 30 per injection.

If the plausibility check 210 is successful the procedure continues with step 211. Here, the dose history recording and/or a calculation of a residual amount of medicament left in the medicament container 21 continues.

With the detector arrangement 98 electrically connected with the machine-readable identification 28 it is possible to permanently monitor the correct installation and/or configuration of the injection device 1 by the add-on device 30. This way, patient safety can be enhanced and unintended or unauthorized use of the injection device 1 can be at least hindered or even blocked.

### REFERENCE NUMBERS

1 injection device
2 distal direction
3 proximal direction
4 dose incrementing direction
5 dose decrementing direction
6 body
7 container part
8 dose dial
9 trigger
10 housing
11 connector
12 needle assembly
13 injection needle
14 inner needle cap
15 outer needle cap
16 protective cap
17 label
18 piston
19 piston rod
20 drive mechanism
21 medicament container
22 barrel
23 seal
24 medicament
25 outlet
26 window
27 dial extension
28 identification
29 electronic device identifier
30 add-on device
34 electronic module
36 printed circuit board
37 reader
38 transceiver
39 transceiver
40 module memory
42 clock
44 module processor
46 power source
48 sensor
50 indicator
51 display
52 signal generator
53 display section
54 display section
60 device body
61 sidewall
62 flange portion
63 receptacle
64 fastening rib
65 alignment feature
66 counter alignment feature
66 neck portion
67 distal end
68 proximal end
69 extension
70 movable part
80 dose setting element
81 drive member
82 volume indicator
83 encoding
85 locking mechanism
86 locking controller
88 identification
89 electronic container identifier
90 electronic circuit
91 antenna
92 memory
93 integrated circuit
94 processor
95 substrate
96 power source
97 adhesive layer
98 detector arrangement
99 RFID tag
100 electronic device
101 housing
103 wristband
110 hand
111 wrist
112 palm
114 thumb
116 finger
120 injection system
137 device reader
138 device transceiver
139 device transceiver
140 device memory
144 device processor
146 device power source
151 device display
152 device signal generator
153 illustration
154 notification
156 indicator
160 receptacle
161 sidewall
162 inside surface
163 end face
164 fastening structure
165 groove
166 stop
170 insert section
171 sidewall
172 outside surface
173 stepped portion
174 counter fastening structure
175 protrusion
180 switch
181 contact spring
182 actuating element
190 electrical circuit
191 conductive wire
192 conductive wire
194 terminal
195 terminal
196 general-purpose input/output terminal
197 resistor
198 capacitor

1-18. (canceled)

19. An injection device for injecting of a dose of a medicament, the injection device comprising:
a housing comprising a body and a container part, wherein the container part is configured to accommodate a medicament container and wherein the container part is detachably connectable to the body,
a drive mechanism arranged in the body and operable to expel the dose of the medicament from the medicament container,
a machine-readable identification on or inside one of the body and the container part,
a detector arrangement on or inside one of the body and the container part and being operable to detect a mechanical connection between the body and the container part,
wherein the machine-readable identification is electrically connected to the detector arrangement and is readable by an electronic module of an add-on device connectable to the injection device and wherein the machine-readable identification is indicative of the mechanical connection between the body and the container part.

20. The injection device according to claim 19, wherein the machine-readable identification and the detector arrangement are electrically connected by an electrical circuit.

21. The injection device according to claim 19, wherein the detector arrangement comprises an electromechanical switch on or in one of the body and the container part and wherein the other one of the body and the container part comprises an actuating element configured to engage the electromechanical switch when the container part is connected to the body and configured to disengage the electromechanical switch when the container part is disconnected from the body.

22. The injection device according to claim 21, wherein the body comprises a fastening structure and the container part comprises a counter fastening structure, wherein the electromechanical switch is provided on the fastening structure and the actuating element is provided in or on the counter fastening structure, and wherein the container part and the body are mutually fastenable to each other through a mechanical engagement of the fastening structure with the counter fastening structure.

23. The injection device according to claim 19, wherein one of the body and the container part comprises an insert section having an outside surface and wherein the other one of the body and the container part comprises a receptacle having an inside surface and sized to receive the insert section and wherein the detector arrangement is provided on the outside surface of the insert section or on the inside surface of the receptacle.

24. The injection device according to claim 19, wherein the body comprises a distal end and an oppositely located proximal end, wherein the machine-readable identification is located nearer to the proximal end than the detector arrangement and/or wherein the detector arrangement is located nearer to the distal end than the machine-readable identification.

25. The injection device according to claim 19, wherein the machine-readable identification comprises an electronic circuit, wherein the electronic circuit comprises an antenna and an integrated circuit operable to wirelessly communicate with the electronic module of the add-on device.

26. The injection device according to claim 25, wherein the machine-readable identification comprises a RFID tag, NFC tag, or UWB tag.

27. The injection device according to claim 25, wherein the machine-readable identification comprises a memory configured to store at least one of a device information, a container information and a use-related data.

28. The injection device according to claim 19, wherein the detector arrangement is configured to enter into a first detector state when the body is connected to the container part and is further configured to enter into a second detector state when the body is disconnected from the container part.

29. The injection device according to claim 28, wherein the machine-readable identification is in a first machine-readable state when the detector arrangement is in the first detector state and wherein the machine-readable identification is in a second machine-readable state, when the detector arrangement is in the second detector state.

30. An add-on device for attaching to an injection device according to claim 19, the add-on device comprising:
a device body fastenable to a portion of the injection device,
at least one of a reader and a transceiver being operable to read the machine-readable identification of the injection device and to generate a reading signal,
an electronic module comprising a module processor coupled to at least one of the reader and the transceiver, wherein the module processor is operable to process the reading signal to determine if the container part is connected to the body.

31. The add-on device according to claim 30, wherein the module processor is configured to sample or to request the reading signal from one of the reader and the transceiver according to predefined time intervals.

32. The add-on device according to claim 30, further comprising a sensor operable to quantitatively determine at least one of a position and a movement of a movable component of the drive mechanism relative to at least one of the housing, the medicament container and the device body and operable to generate a respective sensor signal, and wherein the module processor is operable to determine or to calculate a size of the dose of the medicament, which is set or dispensed by the injection device, on the basis of the sensor signal.

33. The add-on device according to claim 32, wherein the electronic module further comprises a module memory connected to the module processor and operable to store a dosing history, the dosing history including a number of dose sizes dispensed or injected by the injection device and determined or calculated by the module processor.

34. The add-on device according to claim 33, wherein the module processor is operable to determine or to calculate a residual amount of the medicament contained in the medicament container on the basis of the dosing history and on the basis of a connection status between the body and the container part.

35. An injection system comprising
an injection device for injecting of a dose of a medicament, the injection device comprising:
a housing comprising a body and a container part, wherein the container part is configured to accommodate a medicament container and wherein the container part is detachably connectable to the body,
a drive mechanism arranged in the body and operable to expel the dose of the medicament from the medicament container,
a machine-readable identification on or inside one of the body and the container part,
a detector arrangement on or inside one of the body and the container part and being operable to detect a mechanical connection between the body and the container part,
wherein the machine-readable identification is electrically connected to the detector arrangement and is readable by an electronic module of an add-on device connectable to the injection device and wherein the machine-readable identification is indicative of the mechanical connection between the body and the container part, and
an add-on device for attaching to the injection device, the add-on device comprising:
a device body fastenable to a portion of the injection device,
at least one of a reader and a transceiver being operable to read the machine-readable identification of the injection device and to generate a reading signal, and
an electronic module comprising a module processor coupled to at least one of the reader and the transceiver, wherein the module processor is operable to process the reading signal to determine if the container part is connected to the body.

36. The injection system according to claim 35, wherein the add-on device further comprises a sensor operable to quantitatively determine at least one of a position and a movement of a movable component of the drive mechanism relative to at least one of the housing, the medicament container and the device body and operable to generate a respective sensor signal, and wherein the module processor is operable to determine or to calculate a size of the dose of the medicament, which is set or dispensed by the injection device, on the basis of the sensor signal.

37. The injection system according to claim 35, wherein the machine-readable identification of the injection device comprises an electronic circuit, wherein the electronic circuit comprises an antenna and an integrated circuit operable to wirelessly communicate with the electronic module of the add-on device.

38. A method of monitoring operation of an injection device configured for injecting of a dose of a medicament, the method comprising the steps of:
attaching of an add-on device to a portion of the injection device, wherein the injection device comprises a machine-readable identification on or inside one of a body and a container part of a housing of the injection device, and wherein the machine-readable identification is electrically connected to a detector arrangement on or inside one of the body and the container part, wherein the detector arrangement is operable to detect a mechanical connection between the body and the container part,
reading of the machine-readable identification by at least one of a reader and a transceiver of the add-on device and generating a reading signal being indicative of a mechanical connection between the body and the container part, and
determining if the container part is connected to the body by processing of the reading signal.
